(19) United States
(12) Patent Application Publication
(10) Pub. No.: US 2021/0068865 A1
(43) Pub. Date: Mar. 11, 2021

(54) APPARATUSES AND METHODS FOR IMPROVING RECOVERY FROM MINIMALLY INVASIVE SURGERY

(71) Applicant: Noleus Technologies, Inc., Sugar Land, TX (US)

(72) Inventor: Swarna BALASUBRAMANIAM, Sugar Land, TX (US)

(73) Assignee: Noleus Technologies, Inc., Sugar Land, TX (US)

(21) Appl. No.: 17/016,952

(22) Filed: Sep. 10, 2020

Related U.S. Application Data
(60) Provisional application No. 62/898,971, filed on Sep. 11, 2019.
(60) Provisional application No. 62/899,003, filed on Sep. 11, 2019.

(51) Int. Cl. A61B 17/34; A61B 17/00

(52) U.S. Cl. CPC A61B 17/3423; A61B 2017/00831; A61B 17/3431; A61B 17/00234

(57) ABSTRACT

This disclosure relates to apparatuses and methods for preventing the onset of surgical complications and improving patient recovery from surgeries such as mastectomies, herniorrhaphy or hernioplasty. The apparatuses and methods using the apparatuses leads to improved outcomes from chest surgeries to treat hemothorax and pneumothorax. and progression of complications following minimally invasive surgery such as laparoscopic surgery. In one example, a leaf-like polyurethane heat-sealed bilayer that surrounds a plurality of wedge-shaped foam strips that join at a collecting foam portion inside a trocar is subjected to negative pressure provided through silicone tubing which is sealed to the perforated collecting foam portion. Such negative pressure applied for a prolonged period during or after closure of the chest or abdomen laparoscopic surgery, helps prevent fluid loss, abscesses, hematomas, seromas and infection, surgical complications which, in turn, enhances patient recovery, and reduces the length of their hospital stay.

### CROSS-REFERENCE TO RELATED APPLICATIONS

This application claims the benefit of priority to U.S. Prov. App. Nos. 62/898,971 filed Sep. 11, 2019 and 62/899,003 also filed Sep. 11, 2019, and each of which is incorporated herein by reference in its entirety.

### TECHNICAL FIELD OF THE DISCLOSURE

The present invention relates generally to apparatuses and methods for improving post-operative recovery from bowel surgery. More particularly, the present invention relates to apparatuses and methods for preventing the onset and progression of Postoperative Ileus as well as apparatuses and methods for preventing the onset and progression of complications from minimally invasive surgery such as laparoscopic surgery. The present invention also relates to apparatuses and methods for preventing the onset of surgical complications and improving patient recovery from open cavity or open chest surgeries such as brain surgery, mastectomies, herniorrhaphy or hernioplasty. The apparatuses and methods using the apparatuses lead to improved outcomes from chest surgeries to treat hemothorax and pneumothorax.

### BACKGROUND OF THE INVENTION

Postoperative Ileus (POI) is a transient impairment of bowel motility often resulting after abdominal surgery. POI is a common cause in delaying the body's return to normal gastrointestinal (“GI”) function. Despite significant research investigating how to reduce this multi-factorial phenomenon, a single strategy has not been shown to reduce POI's significant effects on length of stay (LOS) and hospital costs. POI is often responsible for extended hospital stays because hospitals will not discharge a patient until after a bowel movement. POI may also be responsible for some post-surgical readmissions to the hospital. As noted by others, the duration of the resulting hospital stay varies with the anatomic location of the surgery, the degree of surgical manipulation, and the magnitude of inflammatory responses. When the surgery directly affects the GI track, the resulting POI is often more severe and takes longer to correct. Traditional treatment of POI includes mobilization, administration of laxatives, open abdomen surgical techniques, and prokinetic agents. Accordingly, there is a need for alternative approaches for treating POI.

Laparoscopy is a type of minimally invasive surgical procedure in which surgery is performed within a patient cavity (such as the abdomen or pelvis) using a laparoscope inserted into the body through a small incision made in the patient's skin. Typically, the laparoscope has a camera and light, which allows internal structures to be seen clearly on an external visual display screen. Laparoscopic surgery, which is also known as keyhole surgery or minimally invasive surgery, allows a surgeon to access and view the internal organs and structure of the body without needing to make large incisions in the skin. In addition to the laparoscope, tubes, probes, small surgical instruments and suction and irrigation sets can be introduced into the body as required using the same or other small incisions.

During a laparoscopy procedure, the abdomen is inflated with a gas (usually carbon dioxide) in order to obtain more easily intelligible images from the laparoscope and also to increase the room inside the patient's body cavities inside which the surgeon can work. During the course of surgery, fluid is pumped into the abdomen to clean the surgical site and suction is used to remove this fluid along with any other bodily fluids and tissue.

A trocar is a medical or veterinary device that is made up of an obturator (which may be a metal or plastic sharpened or non-bladed tip), a cannula (basically a hollow tube), and a seal. Trocars are placed through the abdomen during laparoscopic surgery. The trocar functions as a portal for the subsequent placement of other instruments, such as graspers, scissors, staplers, etc. Trocars also allow the escape of gas or fluid from organs within the body. Complications from laparoscopy include pneumoperitoneum, pulmonary edema and internal hemorrhaging.

Historically, hospital central suction systems, to which a hand-held laparoscopic suction and irrigation is typically connected, are designed for providing relatively high levels of suction (as high as 750 mmHg) over relatively short periods, and are not designed for providing maintained levels of suction for long periods of time.

When surgery requires suction, control buttons on the laparoscopic suction and irrigation set handle are manipulated such that a high suction flow rate is immediately generated under a high vacuum pressure level from the hospital central suction system. The laparoscopic suction and irrigation set does not provide the surgeon with any control over the suction flow rate. Consequently, if the flow rate under suction exceeds the flow rate of medical gas being pumped into the abdominal cavity, the abdomen will start to collapse. This not only has the effect of restricting the surgeon's view of the surgical site, but also limits the length of time the surgeon can use suction and necessitates a period of resting to allow for reinflation of the abdominal cavity. European Patent EP3017833B1 overcomes these limitations by teaching a complicated suction control apparatus for controlling suction flow rate during laparoscopic surgery.

US Patent Publication No. US 2014/0058328 A1 discloses a system and method to vent gas from a body cavity during an endoscopic procedure, in which a vacuum break device has a chamber in fluid communication with an exhaust gas inlet and an exhaust gas outlet, the chamber includes one or more openings in fluid communication with the atmosphere, a body cavity is in fluid communication with the exhaust gas inlet and the exhaust gas outlet is connected directly or indirectly to a suction source.

Vacuum devices have been proposed as a very desirable means of lifting the abdominal wall for creating an operative space within the abdominal cavity. An example of a patent that teaches such a device is U.S. Pat. No. 4,633,865. A significant drawback of the device disclosed by this patent is that when the abdominal wall is lifted by the application of the vacuum, the internal organs within the abdominal cavity rise concomitantly with the upward movement of the abdominal wall. Consequently, an operative space will not be provided or a very minimal operative space will be provided, increasing the risk of iatrogenic injuries.

Mastectomy, breast reduction, breast reconstruction and breast enhancement procedures have become routine cosmetic surgery. In typical surgical techniques for breast enhancement, a silicone or saline filled implant device is inserted into the breast after an incision in locations such as the intramammary fold, or periareolar area. In such procedures, it is often necessary for the surgeon to manipulate the soft tissue of the breast and hold it in place to allow easier access to the skin for a clean incision and placement of the breast implant. This minimizes scarring, provides better aesthetic appeal, and prevents postsurgical complications.

Hernia repair refers to a surgical operation for the correction of a hernia—a bulging of internal organs or tissues through the wall that contains it. This operation may be performed to correct hernias of the abdomen, groin, diaphragm, brain, or at the site of a previous operation. It can be of two different types: herniorrhaphy or hernioplasty.

An operation in which the hernia sac is removed without any repair of the inguinal canal is described as a hernioplasty. Hernioplasty is combined with a reinforced repair of the posterior inguinal canal wall with autogenous (patient's own tissue) or heterogeneous material such as prolene mesh. In contrast is herniorrhaphy, in which no autogenous or heterogeneous material is used for reinforcement.

Normally, the lungs are kept inflated within the chest cavity by negative pressure in the pleural spaces. A lung will partially or completely collapse if air and/or blood collects in the pleural space, thus causing loss of negative pressure (termed pneumothorax and/or hemothorax respectively). Typically, simple pneumothoraces is treated by placing small tubes placed high up on the chest wall. Hemothorax generally requires a device to remove all the blood and bodily fluids that accumulates in the lower portion of the pleural space. The most dangerous type of these conditions is tension pneumothorax (i.e. pressure pneumothorax or valve pneumothorax) and/or, less commonly, tension hemothorax. In this case, the lung not only fully collapses, but the air and/or fluid within the pleural space builds up enough pressure in the chest cavity to cause a significant decrease in the ability of the body's veins to return blood to the heart, which can result in cardiac arrest and death unless treated emergently.

U.S. Pat. No. 7,229,433 describes an apparatus for treating pneumothorax and/or hemothorax that does not require the assembly of parts and can be used by medical personnel with minimal experience and training in treating these conditions. Like conventional chest tubes, such apparatus fail to effectively drain fluids from the chest cavity and also provide no support for post-operative healing and recovery.

Seromas are a frequent complication following surgery, and can occur when a large number of capillaries have been severed, allowing plasma to leak from the blood and lymphatic circulation. Surgical wounds that can lead to seroma formation include wounds resulting from surgery involving an abdominal flap, such as abdominoplasty surgery, breast reconstruction surgery, panniculectomy, and ventral hernia repair.

Conventional surgical drain devices suffer from several deficiencies, particularly when applied following abdominal flap surgery. They fail to drain fluid adequately, are prone to clogging, and fail to promote tissue adhesion within the wound.

### SUMMARY OF THE INVENTION

In view of the aforementioned problems and trends, embodiments of the present disclosure provide apparatuses and methods for improving patient recovery from maximally invasive surgeries that are prone to seromas. preventing the onset and progression of complications from laparoscopic surgery. Aspects of the devices and methods disclosed are described in further detail in U.S. application Ser. No. 15/221,509 filed on Jul. 27, 2016, which is incorporated herein by reference in its entirety and for any purpose.

In another aspect of the disclosure, surgeries such as mastectomies, herniorrhaphy or hernioplasty, a smaller apparatus which can be collapsed and removed from a smaller incision site may be desirable.

In another aspect of the disclosure, apparatuses and methods using the apparatuses leads to improved outcomes from chest surgeries to treat hemothorax and pneumothorax

According to another aspect of the disclosure, an apparatus includes a bilayer encompassing a plurality of foam strips.

In another aspect of the disclosure, a method for preventing the onset and progression complications from laparoscopic surgery includes the steps of placing a trocar with a plurality of foam strips enveloped in a bilayer on the bowels; and applying negative vacuum pressure therapy to the plurality of strips.

In yet another aspect of the disclosure, an apparatus for decreasing post-operative infections or hematoma includes a trocar encompassing a bilayer which, in turn encompasses a plurality of foam strips distributed in a “leaf” pattern adapted for use in minimally invasive surgery such as laparoscopic surgery. This “mini-leaf” patterned foam strips, encompassed in a bilayer design is exuded or deployed from its retracted (or folded or rolled) position by a plunger-like means into a closed chest or abdominal cavity. As already disclosed the foam strips serve as a conduit for removing blood and/or fluid in the post-operative cavity, chest, or abdominal cavity during minimally invasive surgery, wherein the apparatus is fluidly connected to a negative pressure delivery means.

One embodiment for improving post-operative recovery from surgery may generally comprise one or more pliable members having a main stem portion and one or more primary branch portions extending from the main stem portion, one or more layers encompassing the one or more pliable members and having a curved configuration, wherein the one or more layers has a deployed configuration when positioned within a body cavity and a retracted configuration when withdrawn from the body cavity, and a connecting tube in fluid communication with and coupled to the one or more layers at a periphery of the one or more layers, wherein the one or more layers are collapsible into the retracted configuration relative to the connecting tube when a force is applied to the connecting tube.

One embodiment of a method for treating a tissue region may generally comprise advancing a treatment apparatus in a low profile compact shape through an entry lumen into a tissue region to be treated, reconfiguring the treatment apparatus into a deployed and expanded configuration, positioning the treatment apparatus upon the tissue region, applying negative vacuum pressure therapy to the treatment apparatus such that a bodily fluid is removed via the treatment apparatus, and applying a tensioning force to a connecting tube coupled to a periphery of the treatment apparatus such that the treatment apparatus reconfigures into a collapsed configuration about the connecting tube for removal from the tissue region.

Another embodiment for improving post-operative recovery from surgery may generally comprise a pliable member having a main stem portion and at least one primary branch portion extending from the main stem portion, one or more layers encompassing the one or more pliable members, wherein the one or more layers has a deployed configuration when positioned within a body cavity and a retracted configuration when withdrawn from the body cavity, and a connecting tube in fluid communication with and coupled to the one or more layers at a periphery of the one or more layers, wherein the one or more layers are collapsible into the retracted configuration relative to the connecting tube when a force is applied to the connecting tube.

Other aspects of the embodiments described herein will become apparent from the following description and the accompanying drawings, illustrating the principles of the embodiments by way of example only.

### BRIEF DESCRIPTION OF THE DRAWINGS

The following figures form part of the present specification and are included to further demonstrate certain aspects of the present claimed subject matter, and should not be used to limit or define the present claimed subject matter. The present claimed subject matter may be better understood by reference to one or more of these drawings in combination with the description of embodiments presented herein. Consequently, a more complete understanding of the present embodiments and further features and advantages thereof may be acquired by referring to the following description taken in conjunction with the accompanying drawings, in which like reference numerals may identify like elements, wherein:

FIG. 1 is a perspective of an embodiment wherein the apparatus is in situ inside a human;

FIG. 2 is a top view of an embodiment of the present disclosure fully extended in anticipation of placement within the pelvic floor of a patient;

FIG. 3A is a top view of an embodiment of the present disclosure retracted through an approximately 2 cm incision (not shown) and removed from the pelvic floor of a patient (not shown);

FIG. 3B is a perspective sectional view of the creased aspects of present disclosure which assist in retraction and removal;

FIG. 4 is a cross sectional side view of an embodiment illustrating the various components and offset perforation in the polyurethane bilayer;

FIG. 5 is a partial perspective view of the components embodying some aspects of the present disclosure;

FIG. 6 is a top view of an embodiment of the present disclosure similar to FIG. 2 wherein the apparatus is fully extended with tubing which may be connected to a negative pressure means after placement within a patient;

FIG. 7 is a top view of an embodiment of the present disclosure similar to FIG. 2 which were used to determine potential diameter dimensions of a prototype apparatus;

FIG. 8 is a side perspective view of an embodiment of the present disclosure similar to FIG. 7; and

FIG. 9 is a composite of two perspective views of an embodiment of the present disclosure which were used to determine dimensions of a prototype apparatus;

FIG. 10A is a front perspective view of an embodiment of the present disclosure upon deployment in situ inside a maximally invasive, open abdominal surgery in a human in which multiple devices may be deployed at different regions of the body;

FIG. 10B is another perspective of the placement of an embodiment wherein multiple apparatus are in situ inside a patient undergoing abdominal surgery;

FIG. 10C is a front perspective view of an embodiment of the present disclosure upon deployment in situ inside a maximally invasive, open abdominal surgery in a human in which a single device is deployed;

FIG. 10D is another perspective of the placement of an embodiment wherein a single apparatus is in situ inside a patient undergoing abdominal surgery;

FIG. 11A is a front perspective view of an embodiment of the present disclosure showing the apparatus in situ inside a patient in a minimally invasive manner undergoing thorascopic surgery to treat hemothorax;

FIG. 11-1A is a front perspective view of an embodiment of the present disclosure showing the apparatus in situ inside a patient in a maximally, invasive open procedure undergoing surgery to treat hemothorax

FIG. 11B is a perspective of an embodiment wherein the apparatus is in situ inside a human;

FIGS. 12A-12C are a composite of a “fan-shaped” apparatus in various stages of deployment;

FIGS. 12D-12E show a progression view of the “fan” design rolled and loaded into a trocar/cannula apparatus for deployment in minimally invasive/laparoscopic surgery;

FIGS. 13A-13F show one method of operation of any version of the disclosed design into a patient during minimally invasive/laparoscopic surgery;

FIGS. 13-1A-13-1F show another method of operation of any version of the disclosed design into a patient during maximally invasive surgery such as an open abdominal surgery;

FIGS. 14A-14B feature examples of dimensions of another variation of a “leaf” design in an open/deployed state;

FIG. 14C features the apparatus of FIGS. 14A-14B in a “rolled” compact design for use in the trocar/cannula apparatus;

FIGS. 15A-15D show a progression view of the “leaf” design disclosed in

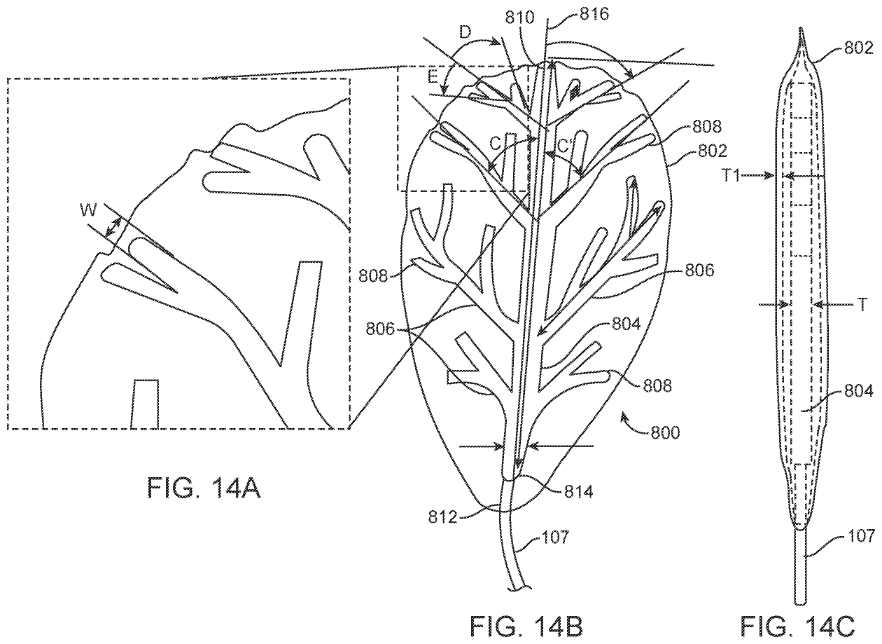

FIGS. 14A-14C rolled and loaded into a trocar/cannula apparatus for deployment in minimally invasive/laparoscopic surgery;

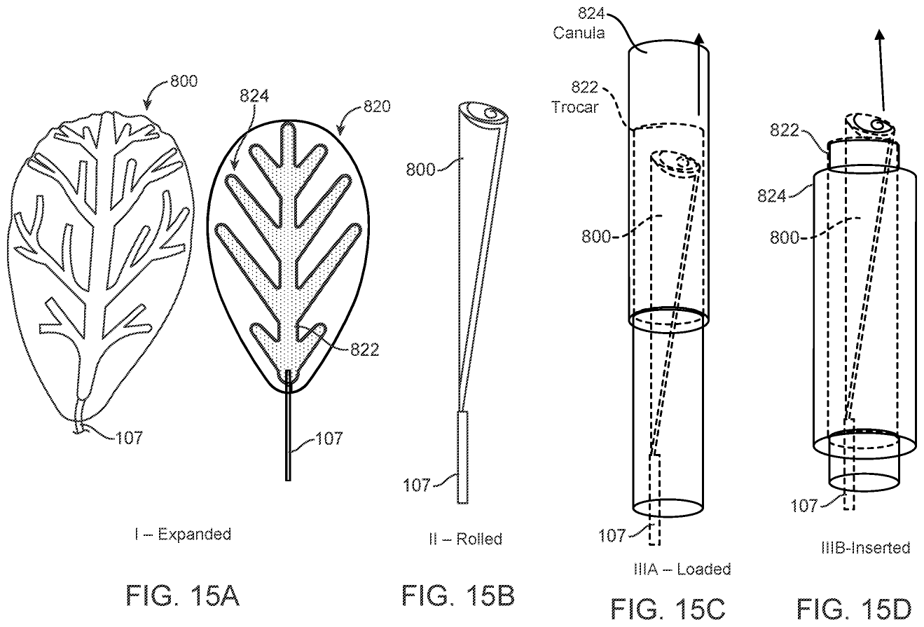

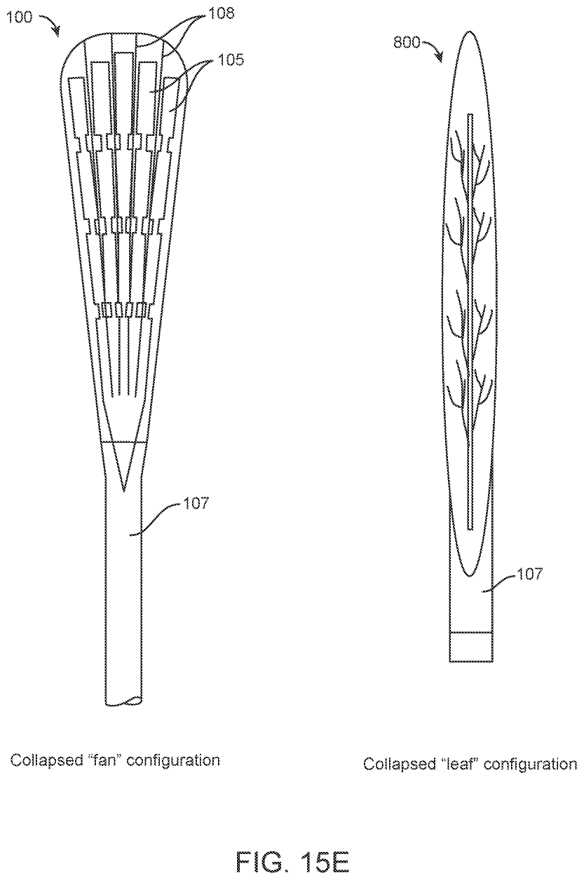

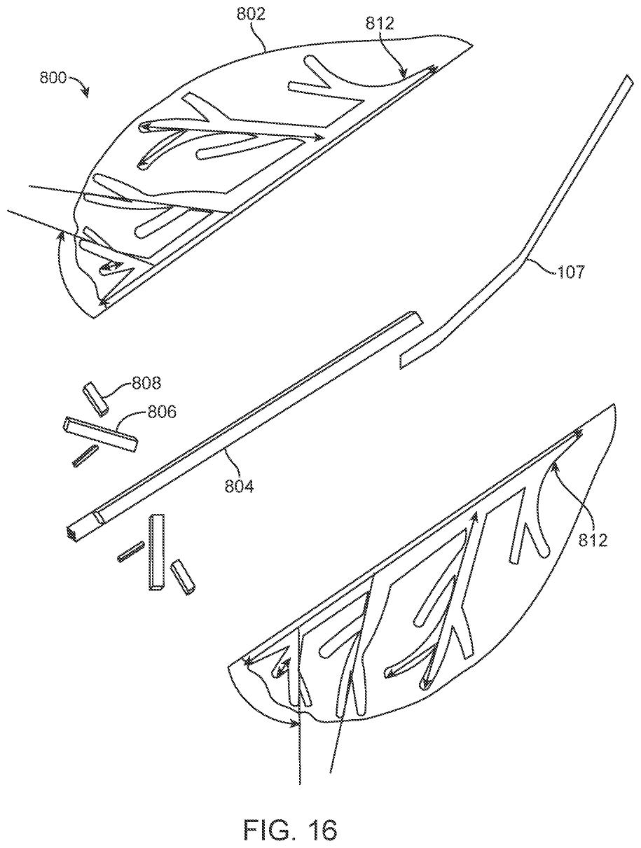

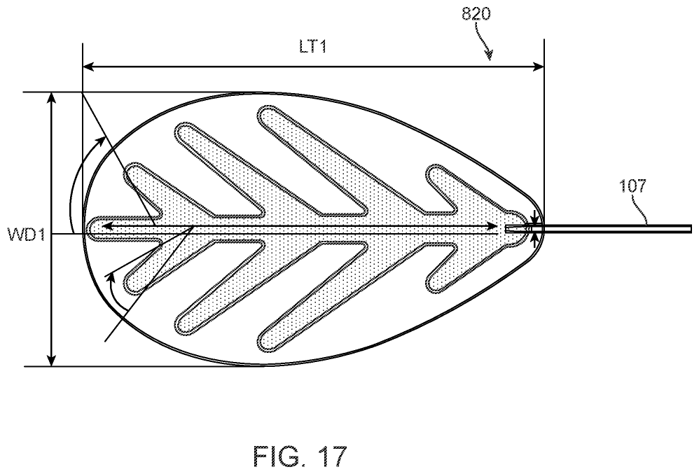

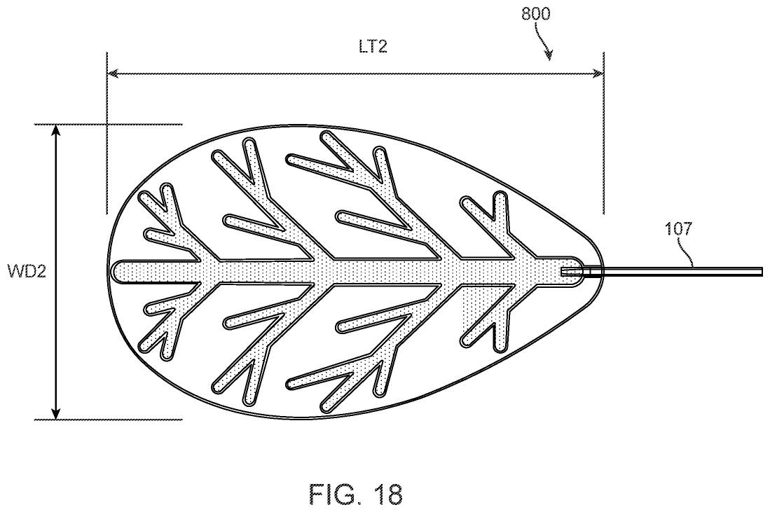

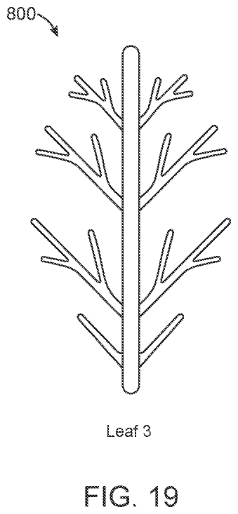

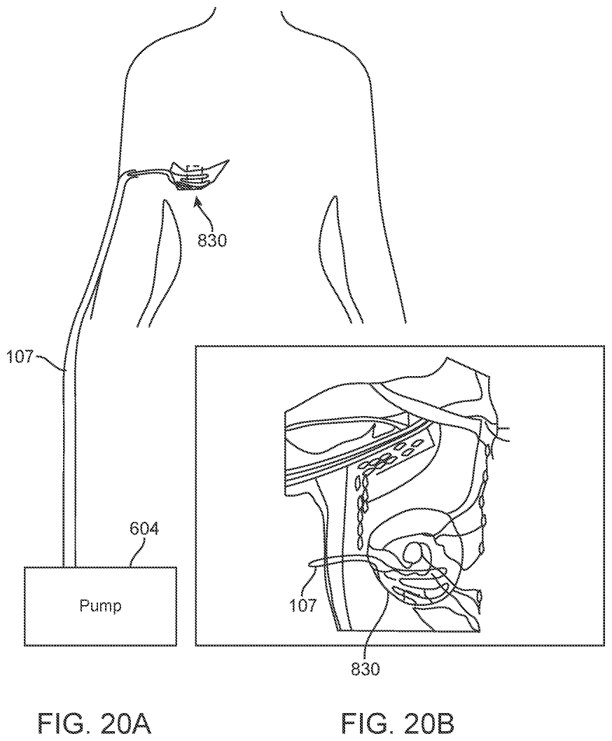

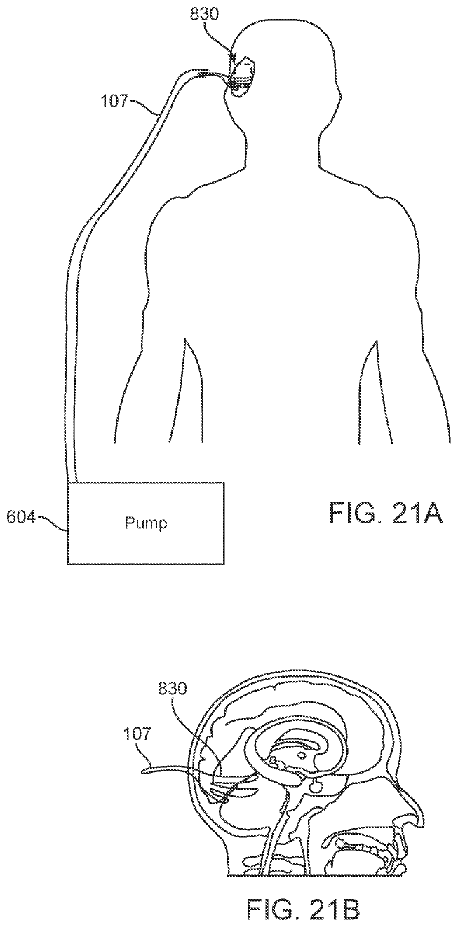

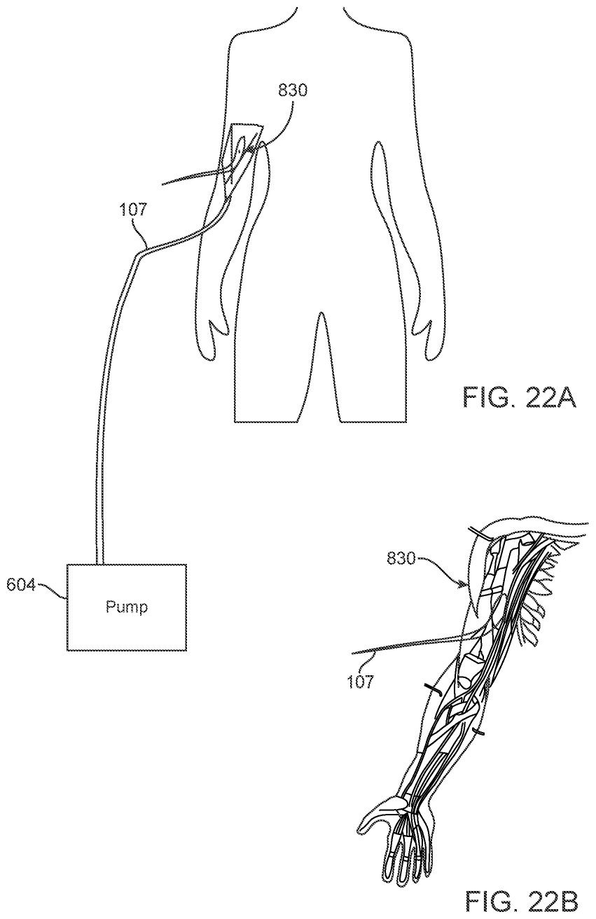

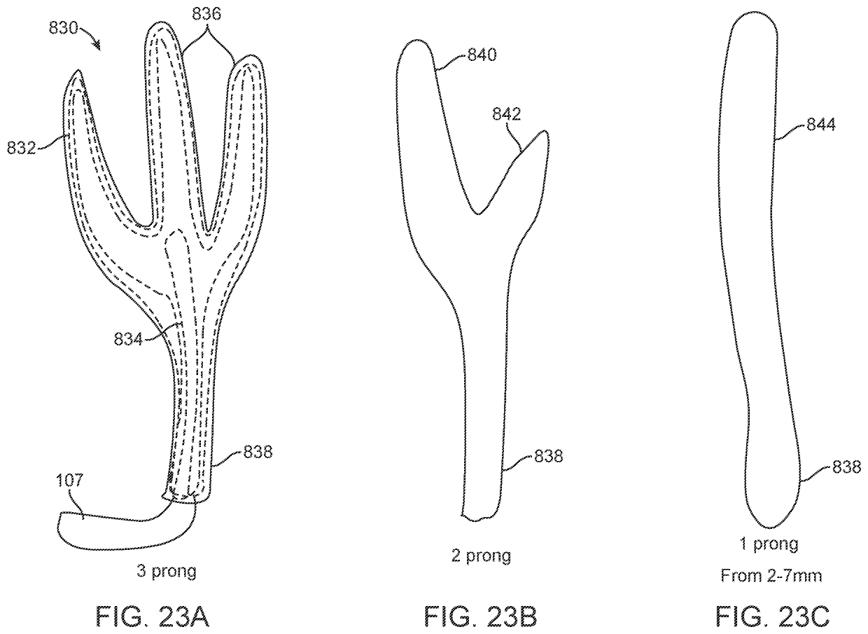

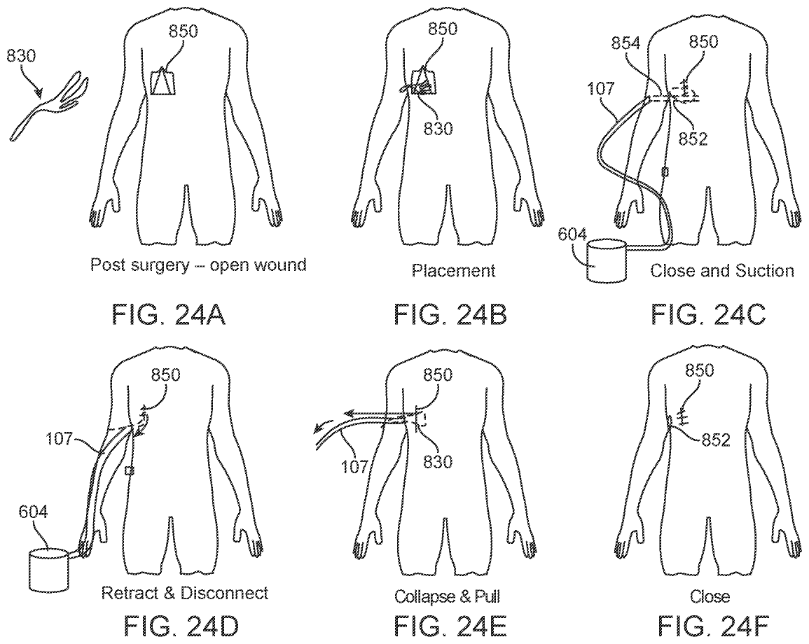

FIG. 15E show a crumpled/collapsed version of the “leaf” design disclosed herein;

FIG. 16 is an exploded view of the components of the “leaf” design;

FIG. 17 is a perspective view of an embodiment of the present disclosure which show examples of dimensions of a prototype apparatus;

FIG. 18 is another perspective view of an embodiment of the present disclosure which show examples of dimensions of a prototype apparatus;

FIG. 19 is yet another perspective view of an embodiment of the present disclosure which were used to determine dimensions of a prototype apparatus;

FIGS. 20A-20B are a front perspective and detail view of an embodiment of the “pitchfork” design of the present disclosure wherein the apparatus is in situ inside a patient undergoing a mastectomy;

FIGS. 21A-21B are perspective views of an embodiment wherein the apparatus is in situ inside a patient undergoing brain surgery;

FIGS. 22A-22B are perspective views of an embodiment wherein the apparatus is in situ inside a patient undergoing surgery for a large wound;

FIG. 23A-23C is a perspective of an embodiment wherein the apparatus is in the “pitchfork” version of the disclosed design; and,

FIGS. 24A-24F show another method of operation of the “pitchfork” version of the disclosed design into a patient during breast surgery or a chest surgery to treat pneumothorax.

### NOTATION AND NOMENCLATURE

Certain terms are used throughout the following description and claims to refer to particular system components and configurations. As one skilled in the art will appreciate, the same component may be referred to by different names. This document does not intend to distinguish between components that differ in name but not function. In the following discussion and in the claims, the terms “including” and “comprising” are used in an open-ended fashion, and thus should be interpreted to mean “including, but not limited to . . . . ”

The term “patient” is used throughout the specification to describe an animal, human or non-human, to whom treatment according to the methods of the present disclosure is provided. Veterinary applications are clearly anticipated by the present disclosure. The term includes but is not limited to mammals, e.g., humans, other primates, pigs, rodents such as mice and rats, rabbits, guinea pigs, hamsters, cows, horses, cats, dogs, sheep and goats. The term “treat(ment),” is used herein to describe delaying the onset of, inhibiting, preventing, or alleviating the effects of a condition, e.g., ileus. The term “donor” or “donor patient” as used herein refers to a patient (human or non-human) from whom an organ or tissue can be obtained for the purposes of transplantation to a recipient patient. The term “recipient” or “recipient patient” refers to a patient (human or non-human) into which an organ or tissue can be transferred.

The term “ileus” as used herein generally refers to partial or complete paralysis or dysmotility of the gastrointestinal tract. Ileus can occur throughout the gastrointestinal tract, or can involve only one or several sections thereof, e.g., stomach, small intestine, or colon. The skilled practitioner will appreciate that ileus can be caused by a great number of factors that include, for example, surgery (e.g., any surgery involving laparotomy, e.g., small intestinal transplantation (SITx); or any surgery involving laparoscopy); intestinal ischaemia; retroperitoneal hematoma; intraabdominal sepsis; intraperitoneal inflammation; acute appendicitis; choecystitis; pancreatitis; ureteric colic; thoracic lesions; basal pneumonia; myocardial infarction; metabolic disturbances, e.g., those that result in decreased potassium levels; drugs, e.g., prolonged use of opiates; and traumas, e.g., fractures of the spine and rib fractures (see, e.g., Oxford Textbook of Surgery, Morris and Malt, Eds., Oxford University Press (1994)). The term also includes post-partum ileus, which is a common problem for women in the period following parturition, e.g., following vaginal delivery (“natural childbirth”) or surgically-assisted parturition. As used herein, the term “post-operative ileus” or POI refers to ileus experienced by a patient following any surgical procedure, e.g., abdominal surgery.

### DETAILED DESCRIPTION OF THE INVENTION

The foregoing description of the figures is provided for the convenience of the reader. It should be understood, however, that the embodiments are not limited to the precise arrangements and configurations shown in the figures. Also, the figures are not necessarily drawn to scale, and certain features may be shown exaggerated in scale or in generalized or schematic form, in the interest of clarity and conciseness. The same or similar parts may be marked with the same or similar reference numerals.

While various embodiments are described herein, it should be appreciated that the present disclosure encompasses many inventive concepts that may be embodied in a wide variety of contexts. The following detailed description of exemplary embodiments, read in conjunction with the accompanying drawings, is merely illustrative and is not to be taken as limiting the scope of the disclosure, as it would be impossible or impractical to include all of the possible embodiments and contexts of the examples in this disclosure. Upon reading this disclosure, many alternative embodiments of the present disclosure will be apparent to persons of ordinary skill in the art. The scope of the disclosure is defined by the appended claims and equivalents thereof.

Illustrative embodiments of the disclosure are described below. In the interest of clarity, not all features of an actual implementation are described in this specification. Any figures are not inferred to be limitations in scope as they are only “example” embodiments of the disclosure. In the development of any such actual embodiment, numerous implementation-specific decisions may need to be made to achieve the design-specific goals, which may vary from one implementation to another. It will be appreciated that such a development effort, while possibly complex and time-consuming, would nevertheless be a routine undertaking for persons of ordinary skill in the art having the benefit of this disclosure.

The return of normal bowel function following any type of surgery is usually a predictable event. The return of the small intestine's peristaltic action begins first, usually 4 to 8 hours post-operatively, and generally becomes complete around 24 hours. The colon resumes its function between 48 and 72 hours postoperatively. However, in some cases, there is a delay or permanent failure of normal bowel function leading to ileus. The pathogenesis of POI is poorly understood, but multiple causes have been suggested: sympathetic reflexes; inhibitory humoral agents; release of norepinephrine from the bowel wall; and the effects of anesthesia agents, opiates, and inflammation.

The surgery can be any surgery that causes and/or puts the patient at risk for ileus. For example, the surgery can involve manipulation (e.g., touching (directly or indirectly)) of the gastrointestinal tract, e.g., the stomach and/or intestines, e.g., small or large intestine (e.g., the colon), and can be any surgery such as that termed generally minimally invasive surgery involving laparotomy or not involving laparotomy (e.g., surgeries involving laparoscopy) or more broadly, the surgery may also include maximally invasive surgery which generally refers to large incisions with create an open cavity in a patient exposing internal organs and tissues to the exterior environment such as open chest, breast, brain, and abdominal surgery any minimally invasive surgery. In certain embodiments, the surgery can be transplant surgery or non-transplant surgery, e.g., surgery involving any organ(s) or tissue(s) in the abdomen, e.g., surgery of the urogenital system (e.g., kidneys, ureter, and/or bladder; and reproductive organs (e.g., uterus, ovaries, and/or fallopian tubes)); the digestive system (e.g., the stomach, small intestine, large intestine (e.g., the colon), appendix, gallbladder, liver, spleen, and/or pancreas); the lymphatic system; the respiratory system (e.g., the lungs); the diaphragm; surgery to treat cancer of any organ or tissue within the abdomen; endometrial surgery; and orthopedic surgeries, e.g., hip surgery.

The treatment of open or chronic wounds by means of applying negative pressure to the site of the wound, where the wound is too large to spontaneously close or otherwise fails to heal, is known in the art. Negative pressure wound treatment (NPWT) systems currently known commonly involve placing a cover that is impermeable to liquids over the wound, using various mechanisms to seal the cover to the tissue of the patient surrounding the wound, and connecting a source of negative pressure (such as a vacuum pump) to the cover whereby an area of negative pressure is created under the cover in the area of the wound.

NPWT promotes the healing of open wounds (such as those that arise during and after surgery) by applying a vacuum through a special sealed dressing. The continued vacuum draws out fluid from the wound and increases blood flow to the area. The vacuum may be applied continuously or intermittently, depending on the type of wound being treated and the clinical objectives. Typically, the dressing is changed several times. The dressings used for the technique include open-cell foam dressings and gauze, sealed with an occlusive dressing or polyurethane which may or may not be permeable, which is intended to contain the vacuum at the wound site. Under certain circumstances, it may be desirable or necessary for NPWT devices and systems to allow delivery of fluids, such as saline or antibiotics to irrigate the wound. The intermittent removal of used fluid supports the cleaning and drainage of the wound bed.

An injury or surgery to the abdomen can result in a wound that cannot be closed straight away. The wound may need to be left open to allow further treatment, or to allow infection to clear. The internal organs, including the bowel, may be left exposed. Sometimes fistulas can form (a fistula is an abnormal passage between either the inside of the body and the skin or 2 internal organs). Open abdomens may be managed in different ways, including using a “Bogota bag” (a sterile plastic bag to contain the bowel), systems with a zip, or dressings. The UK's National Institute for Health and Care Excellence (NICE) concluded that using vacuum therapy to manage open abdomen should be another recommended treatment option for government-provided health insurance such as the UK's National Health Service.

The 7 studies that NICE reviewed involved a total of 5263 patients. Generally, they showed that: Roughly half (45-58%) of patients' wounds could be surgically closed after vacuum therapy compared with rates of 13-78% for other types of temporary dressing. A small number of patients needed an artificial patch to the abdominal wall afterwards—but this also happened after other techniques were used. The proportion of patients who died after vacuum therapy (22-30%) was similar to the number who died after other types of temporary dressing (16-33%). Again, there was no evidence that the deaths were linked to the procedure used.

As already noted, the goals of vacuum therapy are to remove infected material, stop fluid from escaping and help a wound heal. A permeable film, which allows fluid to pass through it, is placed over the wound and a foam sponge or other dressing, discussed further below, such as gauze is placed on top. A drainage tube is placed in the sponge and everything is covered with a transparent sticky film to seal the wound. A small pump then sucks away excess fluid from the wound (the vacuum part of the treatment). A sensing device in the form of a pad placed on top of the foam may be used to make sure that the right amount of suction is used.

Another variant for NPWT is as follows: a dressing or filler material such as foam is fitted to the contours of a wound (which is first covered with a non-adherent dressing film) and the overlying foam is then sealed with a transparent film. A drainage tube is connected to the dressing through an opening of the transparent film. A vacuum tube is connected through an opening in the film drape to a canister on the side of a vacuum pump or vacuum source, turning an open wound into a controlled, closed wound while removing excess fluid from the wound bed to enhance circulation and remove wound fluids. This creates a moist healing environment and reduces edema. This technique is usually used with chronic wounds or wounds that are expected to present difficulties while healing (such as those associated with diabetes).

Such NPWT systems have been commercialized, for example, by Kinetic Concepts, Inc. of San Antonio, Tex., with its proprietary V.A.C.® product line. In practice, the application to a wound of negative gauge pressure, typically involves the mechanical-like contraction of the wound with simultaneous removal of excess fluid. In this manner, V.A.C.® therapy augments the body's natural inflammatory process while alleviating many of the known intrinsic side effects, such as the production of edema caused by increased blood flow absent the necessary vascular structure for proper venous return. As a result, V.A.C.® therapy has been shown to be highly successful in the promotion of wound closure, healing many wounds previously thought largely untreatable. However, treatment utilizing V.A.C.® therapy has been largely limited to open surface wounds. This procedure was approved for reimbursement by the Centers for Medicare and Medicaid Services in 2001.

The second generation system also developed by Kinetic Concepts, Inc. which is commonly used for open abdomen (OA) or laparotomy situations is similar in design to the V.A.C.® product line except for the visceral protective layer (VPL) that contains six foam extensions and provides for improved fluid removal. This ABThera™ OA NPT System uses a non-adherent fenestrated polyurethane, which separates the bowel from the abdominal wall and removes fluid using negative pressure. The ABThera™ Perforated Foam provides medial tension to help minimize fascial retraction and loss of domain. The ABThera™ Visceral Protective Layer provides separation between the abdominal wall and viscera, protecting abdominal contents, which in turn enhances fluid removal. There are no sutures required for placement, which allows for easy removal and replacement. This system has the advantage of faster, more efficient fluid removal as well as enhanced ease of use. However, because of the bulkiness of this system, the abdominal cavity must remain open for the duration of its use. When edema and swelling have been reduced sufficiently, the entire ABThera™ OA NPT System is removed and the abdominal cavity is closed. This may or may not correlate with the patient regaining full bowel function. Thus, there is no apparatus that is intended to prevent POI and help patients recover full bowel function after closure of the abdominal cavity.

The present disclosure teaches apparatuses and methods for improving post-operative recovery from maximally invasive surgeries or surgeries that are prone to seromas. More particularly, the disclosure relates to apparatuses and methods for preventing the onset and progression of Postoperative Ileus. More broadly, the apparatuses and methods may improve outcomes following laparoscopic surgeries.

In one example, a fan-like polyurethane heat-sealed bilayer that surrounds a plurality of foam strips which may be variously shaped, e.g., wedge-shaped, that may join at a collecting portion, such as a foam portion, is subjected to negative pressure provided through a tubing, e.g., silicone tubing, which is sealed to the perforated collecting portion. Such negative pressure applied for a prolonged period after closure of the chest or abdomen, helps prevent fluid loss, abscesses, hematomas and infection, which in turn enhances patient recovery, and reduces the length of their hospital stay. In other examples, the bilayer surrounds a plurality of strips or elements, e.g., wedge-shaped foam strips, which may be distributed in a “leaf” veining pattern that joins at a collecting portion and is subjected to negative pressure. In yet other examples, the bilayer surrounds, e.g., approximately three wedge-shaped strips which may be made of foam and which may be distributed in a pitchfork pattern that joins at a collecting portion.

As disclosed herein, the apparatuses and methods contemplated include those for preventing the onset of surgical complications and improving patient recovery during open chest surgeries such as mastectomies or open cavity surgeries, such as herniorrhaphy or hernioplasty or maximally invasive brain surgery. Generally, the apparatus for decreasing post-operative infections includes a bilayer which, in turn encompasses a plurality of foam strips distributed in a “leaf” pattern adapted for use in maximally invasive surgery, such as, but not limited to, brain surgery, mastectomies, and hernia surgeries.

One differentiating factor in hernia repair is whether the surgery is done open or maximally invasive, or laparoscopically (minimally invasively). Open hernia repair is when an incision is made in the skin directly over the hernia. Laparoscopic hernia repair is when minimally invasive cameras and equipment are used and the hernia is repaired with only small incisions. Such techniques are similar to the techniques used in laparoscopic gallbladder surgery.

Another differentiating factor is whether a mesh is employed or not for treating the hernia. A hernioplasty may be performed with an autogenous material, such as a patient's own tissue, or with a heterogeneous material, such as prolene mesh. Surgical mesh used in hernioplasty is a loosely woven sheet which is used as either as permanent or temporary support for organs and other tissues. The meshes are available in both inorganic and biological materials, and are used in a variety of hernia surgeries. Though hernia repair surgery is the most common application, they may also be used to treat other conditions as well, such as pelvic organ prolapse. Permanent meshes remain in the body, whereas temporary meshes dissolve over time. For example, TIGR® Matrix mesh was fully dissolved after three years in a trial on sheep. Some meshes combine permanent and temporary meshes such as Vipro, a product combining re-absorbable vipryl, made from polyglycolic acid, and prolene, a non-reabsorbable polypropylene.

The disclosed apparatus and methods are particularly suitable for maximally invasive and minimally invasive hernia surgeries, in particular the fan and/or leaf design for abdominal wall hernia surgeries. For example, during abdominal wall surgeries involving removal of tissues and/or large incisions, a great deal of blood and other fluids may accumulate inside a cavity. The operation may involve stitching fascia, adding mesh to “seal” in the hernia(s), and large numbers of stitches along the entire length of an incision. Thus, there is a likelihood of seroma formation which leads to additional surgical complications and even the need to re-operate. The apparatus and methods disclosed herein applied during and after abdominal hernia surgery reduces fluid collection and seromas, thereby reducing surgical complications and improving patient outcomes after surgery. Inguinal (groin) hernias are smaller (and thus do not need NPT therapy in a large surface area) so the “pitchfork” design may reduce the swelling and excess fluid produced during and after such surgeries.

The “mini-leaf”-patterned foam strips, encompassed in a bilayer design, is exuded or deployed from its retracted (or folded or rolled) position into an open cavity such as an open chest or open abdomen. This variant in design allows for insertion into and retraction from the smaller diameter incision which remain after the cavity is closed. As already disclosed the foam strips serve as a conduit for removing blood and/or fluid in any cavity during any surgery, wherein the apparatus is fluidly connected to a negative pressure delivery means.

This “leaf” design may be optimized by reducing the width, depth, other dimensions, number of foam strips, overall shape etc. for a broad range of surgeries. For example, but not meant to be limiting, embodiments for use in the following arenas are contemplated: surgery in the abdomen, hernia surgery, surgery in the thorax/chest region (in which the present disclosure replaces a chest tube to help drain blood or empyema in a pleural cavity), breast surgery (ex: prophylactic mastectomy); thorascopic surgeries (including chest surgery), and brain surgery (using a smaller version of mini-leaf design optimized for extraction from an even smaller incision site).

As disclosed herein, a novel “pitchfork/tubular design” may be used for any surgery prone to seromas. Seromas are prone to occur anytime any tissue is excised leaving an empty space for seroma formation. For example, in both minimally and maximally invasive surgeries, such as breast surgeries, hernia surgeries and surgeries in the arm pit region which are rich in lymph nodes and lymphatic fluids, it is desirable for the tissue flaps generated by surgery to seal to prevent seromas. The application of NPT facilitates the sealing of the tissue flap while concurrently draining fluids from the surgical site. After all fluids have drained out of the closed wound, the pitchfork apparatus is collapsed and extracted from the surgical site.

In the context of open wounds, the methods and apparatus disclosed herein provide additional ant-fluid retention options in the surgical arena.

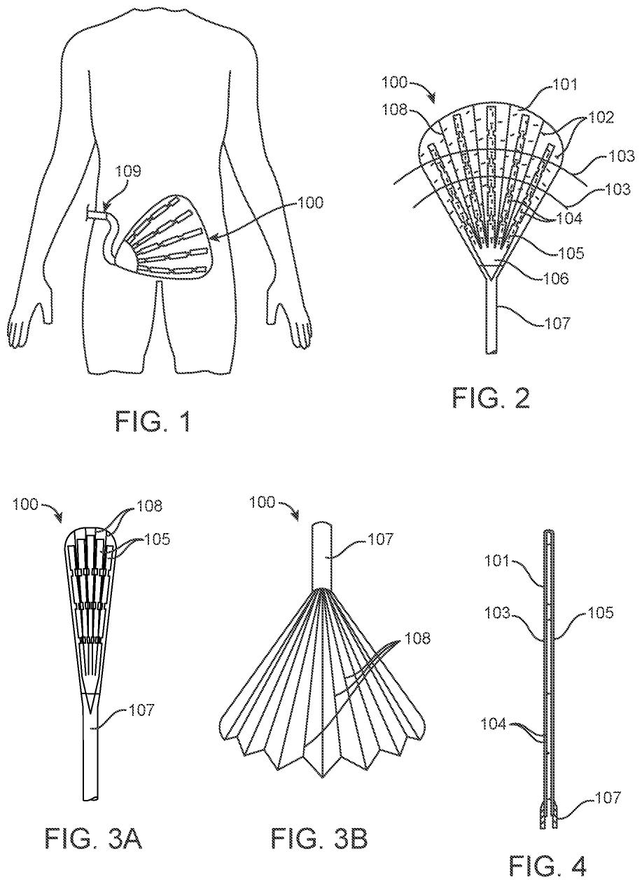

In one embodiment, as shown in FIG. 1, a fan-like polyurethane sealed apparatus 100 such as an enclosure (for example, with heat) such as a bilayer that surrounds a plurality of open cell or porous members, e.g., wedge-shaped foam strips, that join at a collecting portion such as a foam portion, is subjected to negative pressure provided by a tubing which is connected to the collecting portion. Such negative pressure, applied for, e.g., approximately 48 to 72 hours after surgery, reduces complications which, in turn, enhances patient recovery and reduces the length of their hospital stay.

Conventional skin/wound covering materials such as dressings are made up of a bilayer or two layers of material (or film), each layer having specific properties although any number of layers may be used. These conventional dressings for covering cuts, wounds, burns and the like, protect a patient's tissues during the healing process. One layer may include, e.g., a tacky polymer complex layer, for adhesively contacting the skin, which is sealed to a second water vapor-permeable backing layer. The polymer complex layer is produced by mixing together solutions of two hydrophilic polymers which are coprecipitatable, when mixed together, to form a water-insoluble complex. An example of a pair of such polymers is polyacrylic acid and polyethylene oxide.

A modified version of the conventional dressing is used for NPWT. The wound covering used for NPWT typically includes a core layer of a synthetic or semi-synthetic filling, sponge or foam material, such as a cotton gauze or a polyurethane (PU), polyethylene (PE) or polyvinyl alcohol (PVA) sponge which is sealed airtight between two thin polymer (also made of PU, PE or PVA) films, which form a bilayer around the sponge.

The dressing or foam/sponge strips used within the bilayer depends on the type of wound, clinical objectives and patient. For pain sensitive patients with shallow or irregular wounds, wounds with undermining or explored tracts or tunnels, gauze may be used. However, for the present disclosure, foam may be used as it may be cut easily to fit a patient's abdominal space and performs better when aggressive granulation formation and wound contraction is the desired goal.

It should be apparent that while the present disclosure references two dimensional features, the apparatus is three dimensional. As such it is flexible and pliable and intended to be placed around the bowels so as to surround and encompass them within the abdominal cavity. For example, the non-tubing portion of the apparatus 100 in FIG. 1 may be placed at the inlet to the pelvis, for example, almost horizontally or up to, e.g., a 45 degree angle, on the height axis of a supine patient, across the lower abdomen just at the level of the pubis in front and the sacral ala in the posterior.

The apparatus is meant to be a placed temporarily in the abdomen. It is initially placed on the pelvic floor and expanded and flattened over the bowels while the abdomen is open. Cushioned support for the bowels (similar to a hammock supporting a person), is also provided by the apparatus which may enhance patient recovery of bowel function. Placement of the apparatus should be to maximize contact with a large amount of bowel surface area so that negative pressure is applied to most of the surface area of the bowel. Maximizing the surface area interactions between negative pressure and the bowels promotes bowel healing (countering trauma that may arise during and after surgery). By applying a vacuum through a sealed foam bilayer as disclosed herein, the continued vacuum draws out fluid from the bowels and increases blood flow to the area.

As shown in FIG. 1, tubing from the apparatus extends to the outside of the patient's abdomen so that a negative pressure means can be attached. The abdomen is then closed using conventional surgical means known in the art. Once a patient exhibits restoration of bowel function and there is little likelihood of ileus, the apparatus is removed by gently tugging on the tubing portion and pulling it out. The presence of parallel pleats or indentations (element 108 in FIG. 2; not shown in FIG. 1) between the foam wedge-shaped strips facilitates retraction of the apparatus through the approximately, e.g., 2 cm, incision 109 on the patient's abdomen. The “approximately” 2 cm incision 109 may vary by ±10% or by a dimension conventionally used in the surgical art or required for conventional tubing to connect device 100 to any source of negative pressure.

Additionally, while the pleats or indentations 108 are shown to facilitate the collapse or reconfiguration of the device 100 into its low profile, the pleats or indentations 108 may also be omitted from the device 100 which may be allowed to collapse or retract into a low profile in an unrestricted manner. As the tubing 107 is pulled longitudinally, the remainder of the device 100 may collapse longitudinally into its low profile due in part to the location of the tubing 107 being connected along a peripheral edge of the device 100.

For the present disclosure, none of the filler (e.g., foam or sponge strip) material is in direct contact with any viscera or tissue. However, teachings from the analogous art as they relate to filler materials used in conventional wound dressings (which may come into direct contact with viscera and/or tissue), with proven biocompatibility and safety can lead to optimization of the foam strip materials that are often used for the present disclosure. Three types of filler material are used over a wound surface: open-cell foam, gauze and transparent film, or honeycombed textiles with a dimpled wound contact surface. However, other types of filler material may be used in other embodiments and the devices described are not intended to be limited to any particular type of filler material. In general, foam dressings are used to fill open cavity wounds and can be cut to size to fit wounds. The foam dressing is applied, filling the wound and then a film drape is applied over the top to create a seal around the dressing. Open weave cotton gauze can be covered with a transparent film, and a flat drain is sandwiched in gauze and placed onto the wound. The film drape covers the wound and creates a complete seal, and then the drain is connected to the pump via the tubing. It is contemplated that the filler material of the present disclosure includes open cell foam encased in a polyethylene bilayer. However, a single conventional filler material (e.g. only open cell foam) or a combination of other filler materials may be used. It should be noted that the term foam and sponge are used interchangeably.

Companies such as UFP Technologies focus on designing and fabricating dynamic dressings for NPWT that promote and enhance healing as well as expedite the healing process for a patient. Foam is the most commonly used dressing in negative pressure wound therapy because it is easy to apply, suitable for a diverse range of wound types and sizes, and can effectively achieve the goals of NPWT, including a reduction in wound dimensions and improvement in granulation tissue of the wound bed. More specifically, reticulated polyurethane medical foams are used as they are easy to clean, impervious to microbial organisms, and can be made with fungicidal and bactericidal additives for added safety. With open-cell, hydrophobic properties, reticulated foams help evenly distribute negative pressure at the wound site. The pore size of the reticulated foam appears to be a large determinant on the rate of granulation tissue formation. Thus, according to embodiments of the present disclosure, pore size throughout the foam/sponge strips may be manipulated (varied) depending on the particular application. The pore sizes in the reticulated foam also known as open cell foams may be varied depending on the application requirements. These reticulated foams may also be further perforated to generate larger pores (or slits or perforations) which facilitate fluid communication between bowel tissue, each layer of the bilayer, the foam strips and pressure from the negative pressure means.

As noted in a review article by C. Huang et al., the commercial KCI VAC system, uses three general types of foam: black polyurethane ether (V.A.C. GranuFoam, KCI), black polyurethane ester (V.A.C. VeraFlow, KCI), and white polyvinyl alcohol (V.A.C. WhiteFoam, KCI). The traditional polyurethane ether foam is hydrophobic, whereas the polyvinyl alcohol and polyurethane ester foams are more hydrophilic. The polyurethane ester devices are designed for use with instillation therapy. The properties of the traditional polyurethane ether foams are used for wounds with large fluid drainage and for stimulating granulation tissue formation as needed for OA situations. In contrast, the polyvinyl alcohol sponges have been used in cases where the wound tunnels or when delicate underlying structures, such as tendons or blood vessels, need to be protected. Finally, the increased density and smaller pores of the white polyvinyl alcohol based foam helps to restrict ingrowth of granulation tissue, thereby diminishing pain associated with dressing changes and reducing risk when hypergranulation is a concern. Additionally, the foam may be permeated with silver, which provides an effective barrier to bacterial penetration while offering advanced moist wound healing technologies.

Furthermore, in one embodiment the reticulated polyurethane foam is combined with thermoplastic polyurethane (TPU) films which form the previously described bilayer encasing the foam. TPU films are used widely for medical applications because they offer excellent water, fungus and abrasion resistance. They are also soft, breathable, and conformable which help to enhance patient comfort. These semi-transparent TPU films are non-adherent to human tissue, making replacement and removal painless for patients. For example, manufacturers such as Lubrizol Advanced Materials, Inc. (Cleveland, Ohio) produces a variety of TPU films that are strong, flexible, impermeable, biostable and solvent resistant. Thermoplastics, rather than thermoset films are used as they remain pliable which facilitates placement and removal from the abdominal cavity. Pliability is also important as it facilitates maximization of surface areas interactions between the apparatus provided negative pressure and the bowels.

Alternatively, products such as Acticoat® produced by Smith & Nephew, Inc. (Mississauga, Ontario, Canada) may be used for the encapsulated reticulated foam portion of the apparatus. In particular, a rayon/polyester inner dressing core which helps manage moisture level is enveloped in a silver-coated high-density polyethylene mesh bilayer which facilitates the passage of silver through the dressing. The nanocrystalline coating of pure silver delivers antimicrobial barrier activity within 30 minutes—faster than other forms of silver. Acticoat®'s antimicrobial technology is able to produce silver-coated polyethylene films that can release an effective concentration of silver over several days. Thus, as silver ions are consumed, additional silver is released from the dressing to provide an effective antimicrobial barrier. Such silver-based dressing technology delivers fast-acting, long-lasting antimicrobial barrier control which may assist in preventing contamination of the surrounding tissue. Furthermore, this feature may reduce infections contracted during hospitalization caused by “superbugs” such as MRSA. The sustained release of silver also means fewer dressing changes, resulting in less exposure of the tissue bed to the environment. This reduces the risk of infection, further lowering costs to hospitals.

In another embodiment, the bilayer may be composed of a medical grade TPU with each bilayer being from, e.g., approximately 160 to 800 microns in thickness. The fully extended fan-like apparatus may have a radius of, e.g., approximately 30 cm to provide approximately 1,413 cm² of surface area, the reticulated encapsulated foam thickness is, e.g., 10 mm while each polyurethane bilayer has a thickness of, e.g., 160 microns. The shelf life is approximately 3 years at room temperature and all components are sterile and latex free. An example storage temperature range is −20° F. (−29° C.) to 140° F. (60° C.). An example operating temperature range is 50° F. (10° C.) to 100° F. (38° C.) and the altitude range for optimum performance is 0 to 8,000 ft (0 to 2438 m). The dimensions of the contracted or compressed device should be less than 2 cm so that the necessary abdominal incision for retracting the apparatus is similarly a maximum of 2 cm.

The apparatus may be of any shape (circular, square, trapezoid etc.) but for optimal performance the interior foam strips should be distributed in fan-like, leaf-like or pitchfork-like design when fully extended. Examples note that there are very few right angles on the apparatus as a configuration with few or no right angles mitigates difficulties in retraction and removal of the apparatus from a patient's cavity. Thus, all edges (perimeter) of the apparatus are generally rounded and sealed. A means for sealing is the application of heat to the bilayer as this is simple (does not require the application of any additional attachment means) and safe (chemicals attachment such as with glues might harm patients). However, other means of sealing and attachment known in the art are contemplated by this disclosure. Depending on the size of the cavity and level of fatty tissue present, it may be necessary to place more than one of the apparatus within the cavity of a patient, to fully encompass their organ or tissue. In contrast, should the patient have a smaller frame with smaller viscera, the apparatus may be cut to reduce the radius (or size) to accommodate smaller organs/tissues and insertion sizes. As the bilayer is heat-sealable, the present disclosure permits flexibility within the operating room to create an apparatus of variable design that is tailor made for the type of surgery, patient size etc.

In addition, the exact compositions of the bilayer may be modified depending on the application, permeability and desirable flexibility. Additional enhancements to the foam and/or polyethylene/polyurethane or any of the components may be desirable and are contemplated. For example, the foam may incorporate conventionally known radiopaque additives. Thus, if any portion containing foam is left behind in a patient during the retraction process, use of a radiopaque foam can identify this upon x-raying the patient. This reduces patient complications that may arise due to such errors during surgery. Optionally, other luminescent or opaque materials embedded into one or all components of the dressing or other materials may be used to enhance visibility.

As shown in FIG. 2, one embodiment of the apparatus 100 is a fan-like, compressible polyethylene or TPU bilayer 101 enveloping multiple wedged foam or sponge strips 105. The number of sponge strips 105 is five, for this exemplary use, as illustrated in FIG. 2, however this number may be increased or decreased during apparatus manufacturing depending on the optimal sizes of the encased sponge strips. Specifically, if each of the foam strips is broadened, the number of strips would decrease, while a reduction in the surface area of each foam strip may necessitate increasing the number of strips. In any event, it is desirable that the retracted and condensed apparatus be able to be withdrawn from the approximately less than, e.g., 2 cm incision (109 in FIG. 1) through the tubing portion 107 as it exits the abdominal cavity.

There may be randomly spaced perforations 102 on the polyethylene bilayer 101 as shown in FIG. 2. The number of perforations on the entire surface of the apparatus is variable. However, the perforations 102 are sufficiently numerous and sufficiently scattered over the surface of the bilayer 101 as to cover the entirety of the bilayer in a manner such as that shown in FIG. 2, that is to say, such that the bilayer does not have large regions of its surface lacking any perforations 102. The shape and size of each perforation is also variable. In another embodiment, each perforation is less than, e.g., 0.3 cm. Optionally, there are perforations 104 in foam strips 105. These are distinct from the previously mentioned pores that are an inherent feature of conventional reticulated foams. As with perforations 102, perforations 104 are sufficiently numerous and sufficiently scattered over the foam strips 105 as to cover the entirety of their surfaces shown in FIG. 2, in a manner such as that shown in FIG. 2, that is to say, such that the surfaces of foam strips 105 do not have large regions lacking any perforations 104. The perforations 104 in a foam strip 105 extend from one side of the foam to the other, i.e. they go through the foam in the thickness direction (into the page, in FIG. 2). In contrast, while the perforations 102 of each polyurethane or polyetheylene bilayer also extend through each individual polyurethane or polyethylene layer, they do not extend through to the second layer of the bilayer. Thus, the perforations 102 in one layer of the bilayer are offset (in the direction(s) of the length and/or the width of the page, in FIG. 2) from the perforations 102 in the other layer of the bilayer, which permits formation of an airtight or near airtight seal between the two layers of the bilayer. This feature facilitates fluid exchange through the foam strip 105 when negative pressure is applied.

Cut lines 103 may be used to accommodate use of the apparatuses in patients with smaller bowels. As previously stated, the apparatus may be cut to reduce the overall radius (size) of the non-tube portion to accommodate smaller abdominal cavities. The recommended process for reducing the radius involves making a semicircular cut through the broader foam (non-wedge) regions of all of the strips in the apparatus 100, pulling out the excess foam from each of the strips and allowing the polyurethane bilayer to self-seal. It is important that the bilayer be allowed to seal so that no foam comes into direct contact with any tissue, as this could lead to inadvertent attachment of foam to tissue, which would make later removal of the entire apparatus 100 difficult and painful for the patient.

The plurality of wedge-shaped foam or sponge strip portions joins seamlessly to a connecting region 106 which may also be composed of a foam material. Alternatively, the plurality of wedge-shaped foam strips may become narrowed to a smaller width as they taper seamlessly to a connecting region 106 (not shown). The entire plurality of sponge strips enveloped in a bilayer portion (the non-tubing portion) is further sealed by any conventional means to a tube-like extension of silicone 107 which extends across the abdominal cavity to the exterior thereof and is connected to a vacuum source. Sponge strips 105 are illustrated as being parallel (in terms of a polar coordinate system such as would be understood to apply to the partial-circular fan shape of the apparatus 100 as seen in FIG. 2) and extend from near the radially outer end of the fan (i.e., near the circumference if the fan were a circle) to element 106, which lies at/near the radially inward end.

Also, as illustrated in FIG. 2, there may be parallel indentations or creases 108, one (as shown in FIG. 2, or more) disposed between each pair of adjacent foam strips 105 (parallel to the pair), which facilitates the pleating or folding (fan-like) of the apparatus during retraction from the abdomen. The number, radius (i.e., extent, length) and depth of these pleats 108 is variable and may be optimized depending on the number of foam strips 105 present and the dimensions of the foam strips. (Again, the use of the term “parallel” refers here to the above-mentioned polar coordinate system, not a Cartesian coordinate system.)

As previously discussed, while the pleats or indentations 108 are shown to facilitate the collapse or reconfiguration of the device 100 into its low profile, the pleats or indentations 108 may also be omitted from the device 100 which may be allowed to collapse or retract into a low profile in an unrestricted manner. As the tubing 107 is pulled longitudinally, the remainder of the device 100 may collapse longitudinally into its low profile due in part to the location of the tubing 107 being connected along a peripheral edge of the device 100.

The apex (radially inward end) of each foam strip is integrated into a connecting sponge portion as shown in FIG. 2. Once the apparatus is placed in the abdominal cavity so that it supports the bowels, any conventional means to provide negative pressure such as a vacuum pump can be attached to the tubing portion 107 of the apparatus. The connector (not shown) between the vacuum pump tubing and the tubing portion 107 of the apparatus may be a Scienceware® Quick Connector from Bel-Arts Product. The specific components are two barbed polyethylene connectors that assemble tightly together with a male-female center taper. These connectors are specifically designed to be used in connecting and disconnecting vacuum lines and other tubing assemblies which are subject to great variations in pressure.

The pump can be set to deliver continuous or intermittent pressures, with levels of pressure depending on the device used, varying between −125 and −75 mmHg depending on the material used in the foam strips and patient tolerance. Pressure can be applied constantly or intermittently. As with standard negative pressure systems, continuous negative pressure (−125 mmHg) is recommended while pressures below −125 mmHg are not recommended. Pressure can be applied with a conventional medical grade vacuum pump or in emergency situations, any source of vacuum such as a portable hand-held suction pump.

This effects a pulling together of the tissue/wound edges and draining of excess fluid. Furthermore, “micro-massage effects” (also known as “microstrain” effect) may enable cell growth and stimulation of new tissue formation.

FIG. 3A depicts the apparatus of FIG. 2 in its retracted or contracted state. As shown, indentations 108 facilitate the fan-like “folding” of the apparatus to reduce the overall dimensions of the apparatus and permit retraction of the apparatus from the approximately 2 cm incision to the exterior of the abdomen. As the abdominal cavity is closed after placement of the apparatus, this retract-ability feature obviates the need for additional surgery to remove the apparatus following post-operative recovery.

FIG. 3B is a perspective sectional view of apparatus 100 viewed from the radially outward edge thereof. FIG. 3B shows the creased aspect of apparatus 100, which assists in retraction and removal of the apparatus from the abdomen. Indentions 108 in the non-tube-like portion assist in the folding and removing of the apparatus by simply tugging on the tube-like portion 107 which is dangling from the 2 cm excision. As previously noted, the presence of parallel pleats or indentations on either side of a foam strip facilitates retraction by making it easier for the apparatus 100 to “fold up”. This is similar to the functioning of a foldable fan so that a non-surgeon may remove it from the now closed abdomen of a patient when normal bowel function has returned (approximately 48 to 72 hours after abdominal closure).

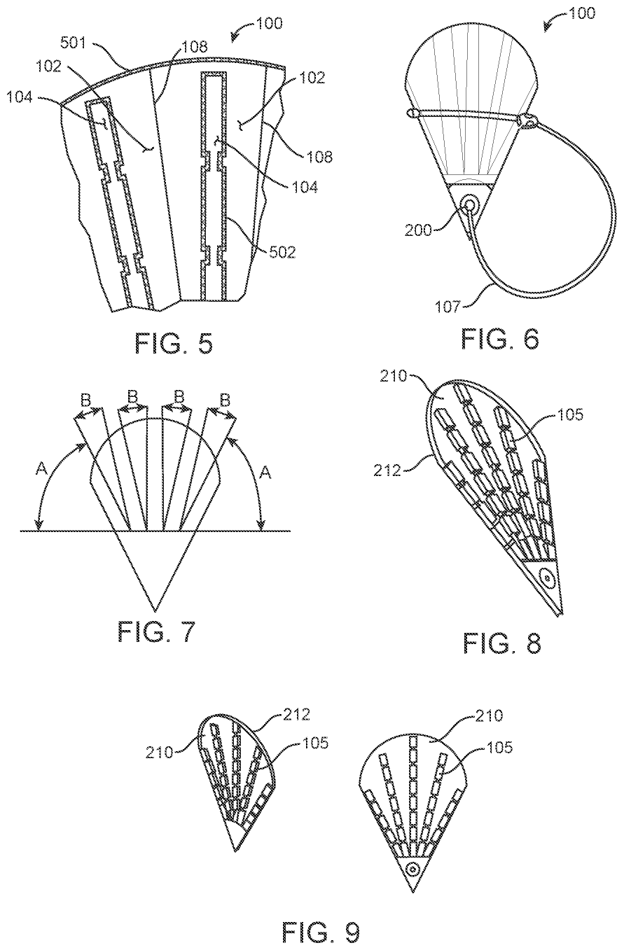

FIG. 4 is an exploded cross sectional side view of an embodiment illustrating the offset perforations 104 in the polyurethane or polyethylene bilayer 101; the heat sealing of the bilayer at the exterior radial end of the apparatus (described further in FIG. 5) and the fusion of the silicone tubing 107 to the bilayer. As discussed, the bilayer 101 encompasses numerous foam or sponge strips 105 disposed within it. As also discussed, optionally, depending on patient's bowel size, the radius (size) of the entire apparatus 100 may be reduced by cutting along cut line 103 (see above, FIG. 2). As seen in FIG. 4, the perforations 104 in bilayer 101, one in the upper layer and one in the lower layer (i.e., “upper” and “lower” as shown in FIG. 4), are offset from each other in radial direction of the apparatus 100 (which is the left-right direction in FIG. 4, and the top-bottom direction in FIG. 2).

Exemplarily, the tubing (or tube-like portion) 107 is silicone and biocompatible but may be made of any material known in the art. As shown in FIG. 4, the tubing 107 may be pre-fused to one or both layers of the sealed (not shown) polyurethane bilayer. It may be, e.g., approximately 24 inches long to facilitate extending across the abdominal cavity to exit from an approximately 2 cm incision (109 in FIG. 1). This tubing portion 107 is, optionally, non-detachable from the rest of apparatus 100 as this protects against leakage of fluid at the connection point between the silicone tubing and the polyethylene bilayer. Alternatively, it is contemplated that the tubing portion 107 be detachable from the apparatus 100 so as to maximize portability and adaptability as shown in FIG. 6. The tubing 107 may be releasably coupled via a connector 200 which may be optionally detached from the apparatus 100.

Another aspect of the present disclosure as illustrated in FIG. 5, is the use of heat sealing to join together the two layers of the bilayer 101. The sheets or layers of the bilayer 101 are sealed, e.g., via heat sealing, adhesives, etc., along their entire (common) perimeter 501 to ensure structural integrity especially during the stress of compression and removal of the apparatus from the abdominal cavity. This sealing also provides the airtight seal mentioned above, that facilitates removal of fluid through the perforations 104 in the wedge foam strips 105 by the vacuum pressure. (While FIG. 5 shows only a portion of heat-sealed perimeter 501 of apparatus 101, it is understood and also seen in FIG. 2 that the entire perimeter 501 is heat-sealed. Note while FIG. 2 shows perimeter 501 it is not labeled with a reference numeral.) Sealing is also used along the periphery 502 of each wedge-shaped foam strip 105, and the seal is contoured to fit the shape of the foam piece. This serves the dual purpose of sealing each of the layers of polyurethane around each foam strip as well as facilitating removal of the foam if a semicircular cut is required to reduce the radius/size of the entire apparatus 100. After a cut is made, the foam core may be left “floating” in the polyethylene bilayer without the seal. This would increase the likelihood of the foam coming into contact with patient tissues which increases likelihood of infection and pain during apparatus removal. Optionally, there may be additional heat sealing to improve overall apparatus integrity.

The narrowing of the foam strips at regular intervals (to form the wedges along each strip) may reduce the weight and overall dimensions of the foam strips. This narrowing is seen in FIG. 5 (two narrowed wedge regions at different radial locations in each strip 105) and in FIG. 2 (four narrowed wedge regions at different radial locations in each of the three central strips 105, and three narrowed wedge regions at different radial locations in each of the rightmost and leftmost strips 105,) However, it is also contemplated that no wedge-like regions within the reticulated foam strips are present to facilitate ease of manufacturing.

In prototyping an embodiment of the present disclosure, various materials were utilized. The attachment means for the tubing 107 that delivers negative pressure may be integrated into the apparatus of the present disclosure as in FIG. 2. Alternatively, as illustrated in FIG. 6, the tubing may be securely attached separately by any means known in the art. It is desirable that the tubing be connected to a stand-alone negative pressure device with a pressure regulator (not shown) after placement within a patient. However, any means for providing negative pressure with or without a means to regulate the pressure (e.g. a vacuum line) may be used. In FIG. 6, the tubing 107 was silicone rubber tubing with a 0.126 in. wall thickness but any dimension of tubing of any length, width, and diameter is contemplated by this disclosure.

FIG. 7 is a top view of an embodiment of the present disclosure similar to FIG. 2 which were used to determine potential diameter dimensions of a prototype apparatus. As illustrated, an angle A, e.g., 63.44° , may be defined between a transverse axis of the apparatus 100 and the outermost strips while an angle B, e.g., 13.28°, may be formed between each adjacent strip.

FIG. 8 is a side perspective view of an embodiment of the present disclosure similar to FIG. 7. In this embodiment, two sheets of film 210, 212, e.g., TPU film from McMaster-Carr (Douglasville, Ga.) that was 0.015 in. thick was used to create a bilayer around open cell foam of ¼ in. thickness. In yet another embodiment, two sheets of 0.015 in. thick TPU film from McMaster-Carr was used to create a bilayer around open cell foam of ½ in. thickness (not shown). FIG. 9 is a composite of two perspective views of an embodiment of the present disclosure which were used to determine dimensions of a prototype apparatus. In this example, the apparatus 100 may have an overall length of 18 in. and an overall width at its widest point of 12 in.

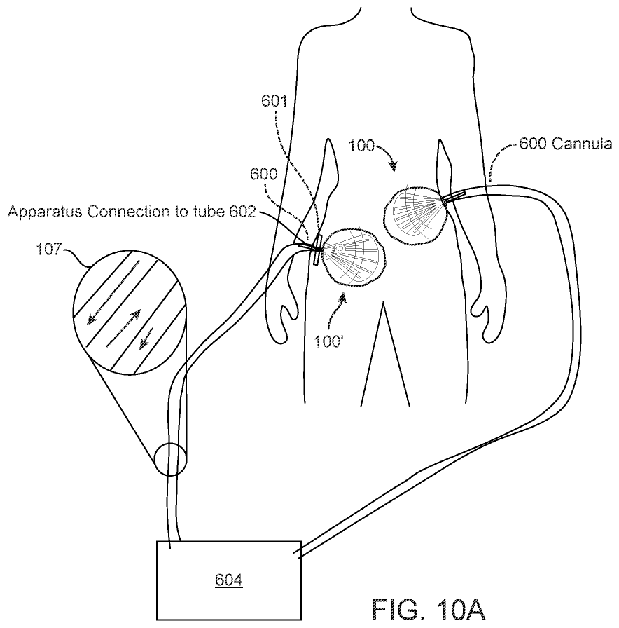

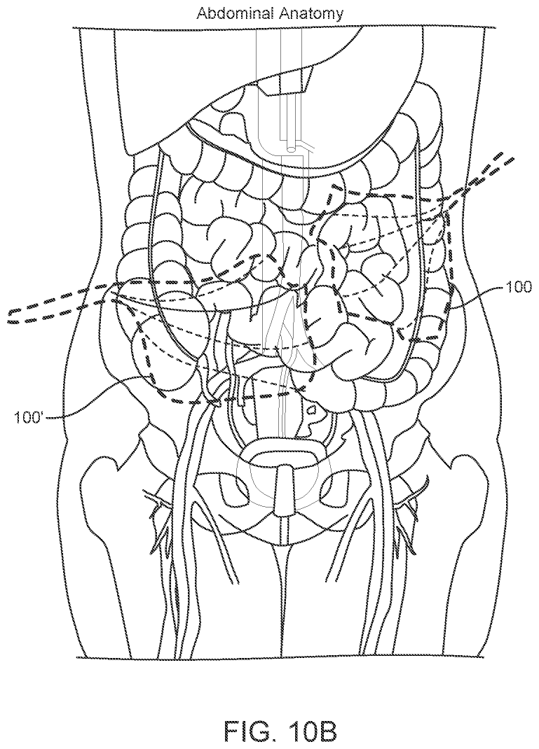

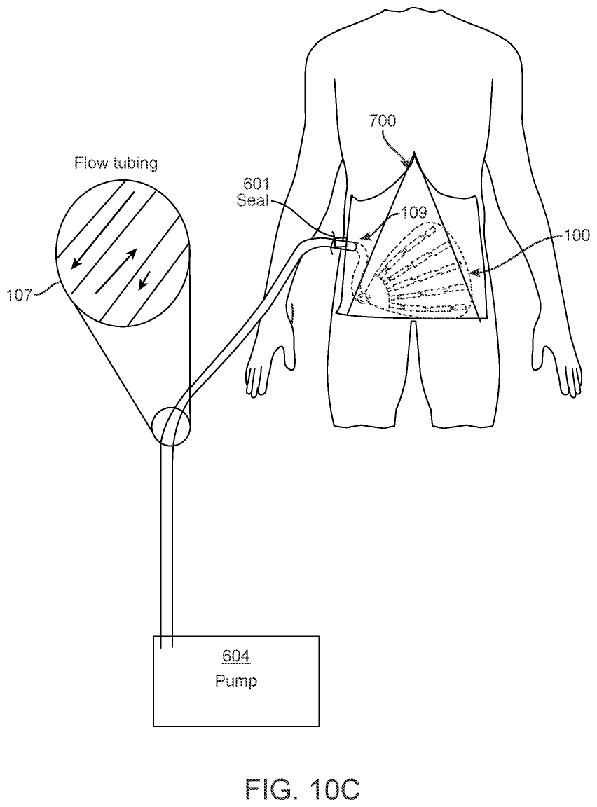

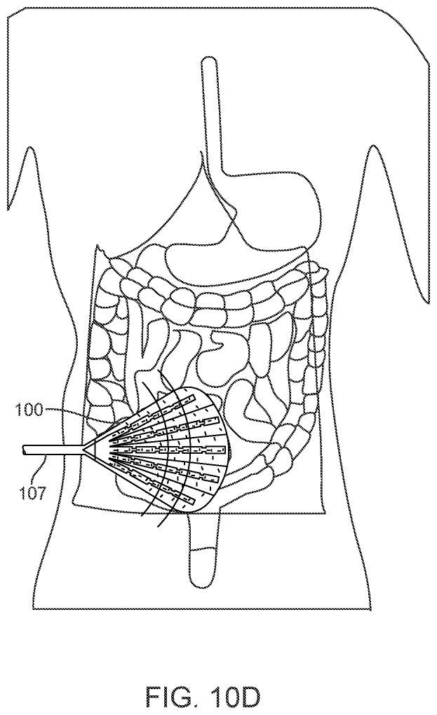

FIG. 10A is a front perspective view of an embodiment of the present disclosure similar to FIG. 7 upon deployment in a minimally invasive surgery. This example illustrates how multiple apparatus 100, 100′ may be used simultaneously in different regions of the body in a minimally invasive manner. This example also illustrates how one or both apparatus 100, 100′ may be advanced into the body cavity in its low profile configuration through a cannula 600 and into the body cavity for minimally invasive access. The apparatus 100, 100′ may be configured into their low profile during insertion and advancement through the respective cannula 600 and once within the body cavity, the apparatus 100, 100′ may be reconfigured into its expanded configuration for placement upon the desired tissue region. The cannula 600 may each incorporate a seal 601 to prevent leakage of gas or fluid from within the body, e.g., to maintain pneumoperitoneum. The tubing 107 coupled to the apparatus 100, 100′ may be fluidly coupled to a pump 604 via an apparatus connection 602 and the tubing 107 may be used to remove the collected fluid during treatment, as well as optionally provide an inflation gas through the cannula 600.

Multiple apparatus 100, 100′ may be used depending on circumstances for minimally invasive/laparoscopic surgery. While the figure depicts multiple apparatus being controlled by vacuum delivered through a single pump, it is contemplated that any combination and number of apparatus connected to any combination and number of pumps and/or controllers is feasible.

FIG. 10B is another perspective illustrating how the apparatus 100, 100′ may be placed in situ inside a patient undergoing, e.g., abdominal surgery, in laparoscopic hernia surgery in a minimally invasive procedure.

FIG. 10C shows a front perspective view of another embodiment during a maximally invasive, open abdominal surgery where an incision 700 may provide open access to the body region of interest for treatment. The apparatus 100 may be positioned upon the tissue region, as shown, while the tubing 107 may be passed through a separate incision 109 or through a portion of the larger incision 700 while the tubing 107 may be maintained through the body with a seal 601. Once the treatment has been completed and the apparatus 100 desirably situation upon the tissue, the incision 700 may be closed with the apparatus 100 remaining within the patient body.

FIG. 10D is another perspective illustrating how the apparatus 100 may be placed in situ inside a patient undergoing, e.g., abdominal surgery, in a maximally invasive, open procedure.

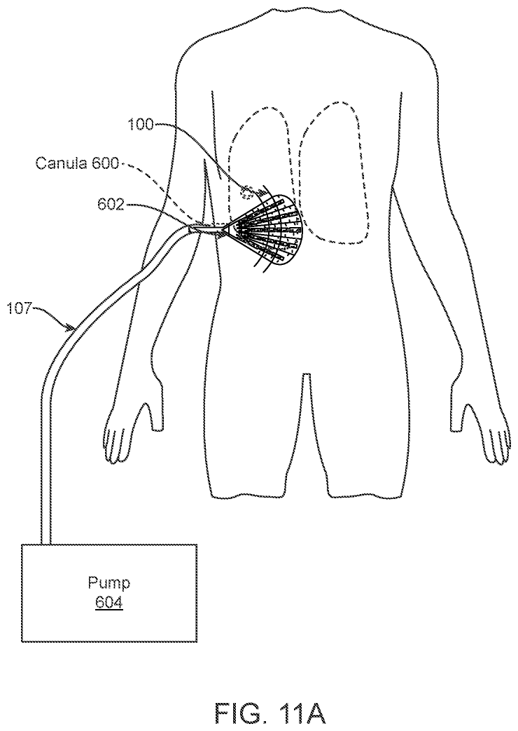

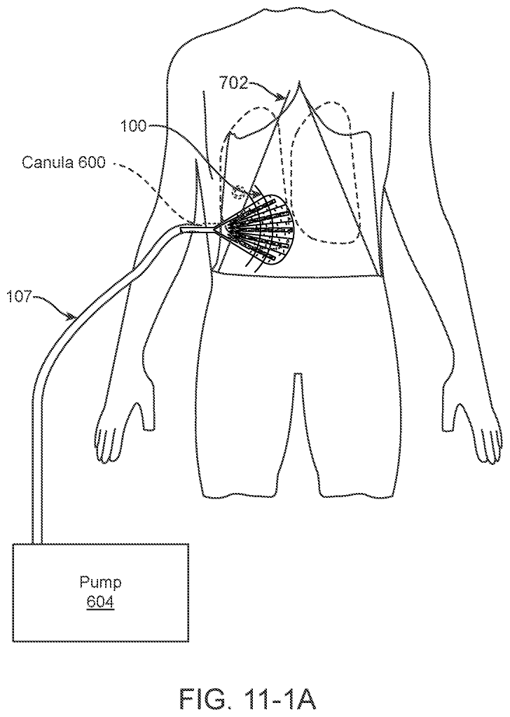

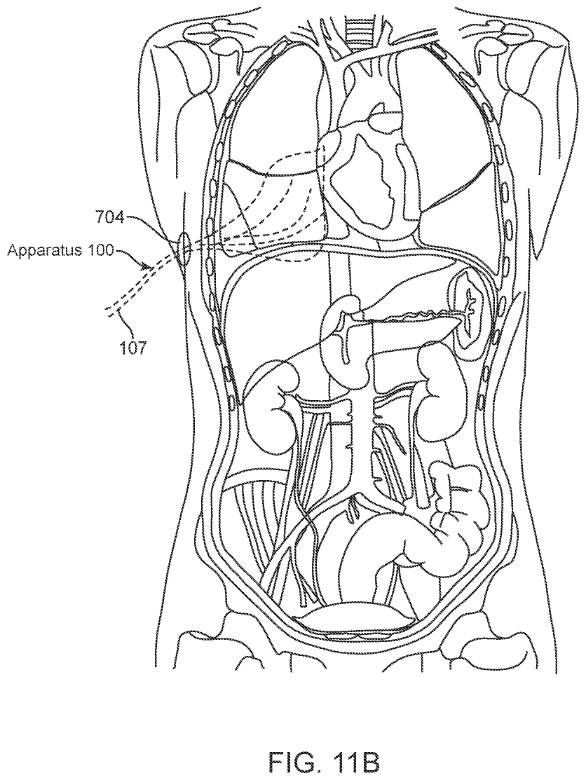

FIG. 11A is a front perspective view of another embodiment where the apparatus 100 may be deployed in a minimally invasive chest surgery through a cannula 600 for thorascopic surgery to treat, e.g., hemothorax.

FIG. 11-1A is a front perspective view of another embodiment where the apparatus 100 may be deployed in a maximally invasive, open chest surgery through an incision 702.

FIG. 11B is another perspective of the placement of the apparatus in situ inside a patient undergoing thoracoscopic surgery to treat hemothorax. The apparatus 100 may be advanced through an incision 704 and cannula 600 to access the thoracic cavity minimally invasively. Hemothorax is the collection of blood in the thoracic cavity. It occurs when chest trauma, such as rib fracture(s) are significant enough to damage any of the vascular structures in the thorax. As the thoracic cavity fills with blood, the lung has a decreased ability to expand normally, thereby decreasing oxygenation and ventilation. If a hemothorax continues to worsen, death may occur by exsanguination or hypoxia. Also, if the blood within the pleural cavity is not removed, it will eventually clot. This clot tends to stick the parietal and visceral pleura together and has the potential to lead to scarring within the pleura, which if extensive leads to the condition known as a fibrothorax.

Unlike conventional chest tubes/drains, embodiments of the apparatus described herein may be used to treat hemothorax and pneumothorax during and after minimally invasive surgery or open chest, thorascopic surgery by rapidly and efficiently draining blood and other fluids from the chest cavity. Because of the larger, flatter surface area of the bilayer encompassing multiple draining foam strips, the designs of the present disclosure provide superior drainage which concurrently promotes faster lung healing from chest trauma. It is also contemplated that the apparatus may be used for other purposes during laparoscopic or thoracoscopic surgery to treat hemothorax and pneumothorax.

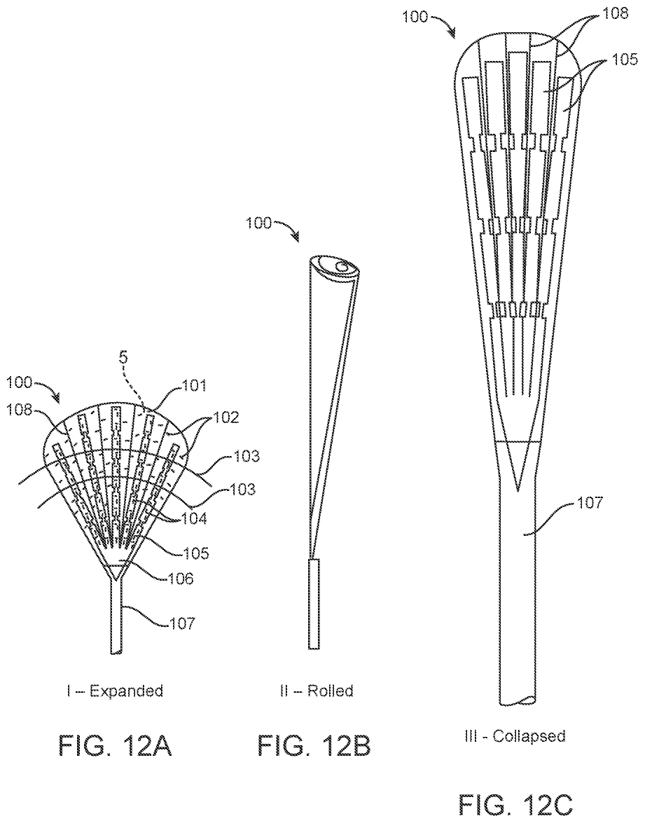

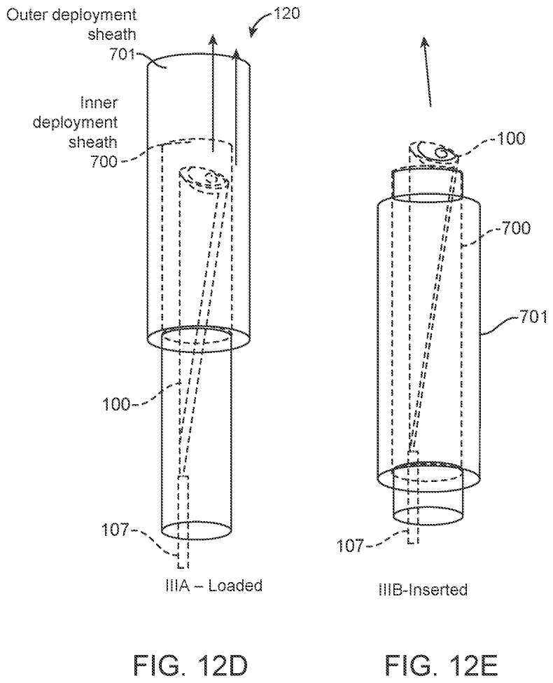

FIGS. 12A-C show composite views of a “fan-shaped” apparatus 100 in various stages of deployment or reconfiguration. FIG. 12A illustrates the apparatus 100 in its fully expanded configuration, e.g., when deployed upon a tissue region for treatment and FIG. 12B shows on variation of the apparatus 100 in a rolled configuration in which the apparatus 100 may be rolled into a longitudinally wound configuration for advancing through a cannula during a minimally invasive procedure. In this manner, the rolled configuration may be advanced through the cannula while remaining in a low profile compressed configuration until the apparatus 100 is advanced past the cannula opening and into the body cavity. Once free of the cannula, the apparatus 100 may unfurl automatically into its deployed configuration for placement upon the tissue region. FIG. 12C illustrates a low profile collapsed configuration which may be utilized when the apparatus 100 is ready for removal from the patient body. The tubing 107 may be pulled or tensioned such that the apparatus 100 collapses about a longitudinal axis coincident with the tubing 107 such that the apparatus 100 may be pulled to collapse for removal through an incision in the patient body for removal from the body cavity, as described herein.

FIGS. 12D and 12E illustrate an example of how the apparatus 100 may be prepared for advancing through a cannula when used in a minimally invasive procedure. Once the apparatus has been rolled into a low profile configuration, as shown, an inner deployment sheath 700 may be positioned over the rolled apparatus 100 to maintain its rolled configuration. The inner deployment sheath 700 and apparatus 100 may then be advanced together 120 into an outer deployment sheath 701 and the assembly may be advanced through a cannula for insertion into the body cavity. Once advanced into the body cavity, the apparatus 100 may be advanced past the inner and outer deployment sheaths 700, 701 to unfurl within the body cavity for placement upon the tissue region for treatment. Alternatively, the inner deployment sheath 700 and apparatus 100 may be advanced simultaneously through the outer deployment sheath 701 which may remain through the cannula or directly through an incision such that the inner deployment sheath 700 and apparatus 100 are positioned within the body cavity in proximity to the tissue region to be treated. The apparatus 100 may then be advanced distally beyond the inner deployment sheath 700 or the inner deployment sheath 700 may be retracted to expose the apparatus 100 for deployment.

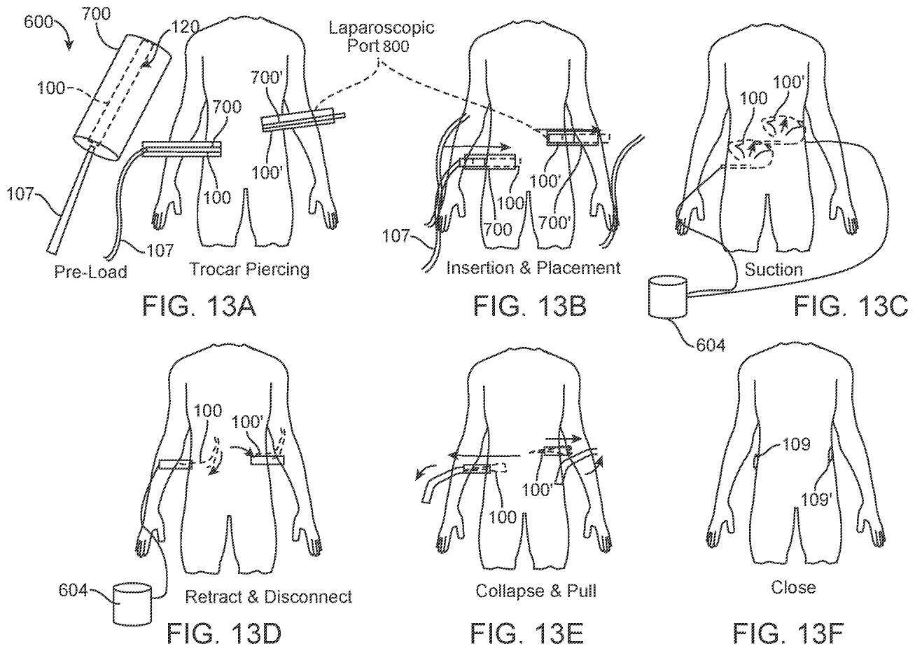

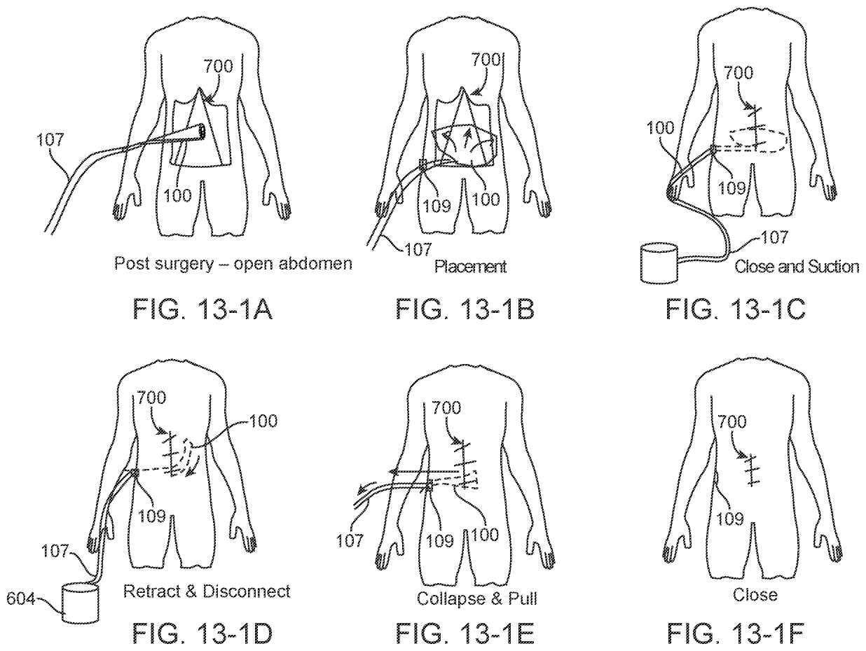

FIGS. 13A-13F illustrate one method of operation of any version of the disclosed design into a patient during minimally invasive surgery. In this example, the apparatus and inner deployment sheath 700 may be configured for its low profile rolled configuration and one or more corresponding trocars may be used to create openings into the patient body, as shown in FIG. 13A, through which any number of laparoscopic procedures may be accomplished. Once the surgical procedure has been completed, the apparatus 100 and inner deployment sheath 700 may be advanced through the incision for accessing the body cavity. After insertion, the apparatus is uncompressed in the region of surgery by means of a plunger-like assembly so as to be deployed for placement around an organ or tissue. The deployed apparatus is left within the body to continue NPT in situ. In this variation, two devices 100, 100′ are shown being inserted through a respective incision for deployment, as shown in FIG. 13B, although a single device may be used or more than two devices may also be utilized.

With the apparatus 100, 100′ deployed and positioned upon the tissue within the body cavity, suction may be applied via a pump 604 fluidly coupled to both apparatus 100, 100′, as shown in FIG. 13C, for draining any bodily fluids and reducing swelling of the tissue. Alternatively, each apparatus may utilize its own individual pump. Once the treatment has been completed, the pump 604 may be disconnected from each apparatus 100, 100′, as shown in FIG. 13D, and the apparatus may each be removed by tensioning or pulling upon the respective tubing such that the apparatus collapses into its collapsed configuration for removal through each respective incision 109, 109′, as shown in FIG. 13E. With the devices removed, the incisions 109, 109′ may be closed, as shown in FIG. 13F.

FIGS. 13-1A-13-1F illustrate another method of operation of any version of the disclosed design into a patient during maximally invasive surgery such as an open abdominal surgery. With an incision 700 for accessing, e.g., the abdominal cavity, the apparatus 100 may be similarly configured into its low profile rolled configuration, as shown in FIG. 13-1A, and inserted either through a separate incision 109 or placed through the incision 700 such that the tubing 107 extends away from the patient body after the completion of a surgical procedure. In either case, the apparatus 100 may be unfurled into its deployed configuration for placement upon the tissue region, as shown in FIG. 13-1B. With the apparatus 100 deployed upon the tissue, the incision 700 may be closed while the tubing 107 remains fluidly coupled to the apparatus 100 while extending, in this example, through incision 109, as shown in FIG. 13-1C.

Once any bodily fluid has been sufficiently drained and tissue swelling reduced, the tubing 107 may be disconnected from the pump 604 and the tubing 107 may then be tensioned or pulled such that the apparatus 100 apparatus collapses about the tubing connection on the periphery of the apparatus 100, as shown in FIGS. 13-1D and 13-1E. The collapsed apparatus 100 may be retracted through the incision 109 while maintaining tension on the tubing 107 until the apparatus 100 has been completed removed from the body. The remaining incision 109 may be then closed, as shown in FIG. 13-1F.

FIGS. 14A-14C show front, detail, and side views of another example of the apparatus which is configured to have a shape similar to a leaf where the enclosure or layers 802 are shaped in a curved obovate or oval configuration. The periphery of the apparatus 800 curves gently from a proximal end at a tubing connection 812 where the tubing 107 is coupled to the apparatus 800 and curves outwardly in the obovate or oval configuration to form a gentle radius at a distal end of the apparatus 800. The apparatus 800 may be symmetrically shaped along its length about a longitudinal axis 816 while the enclosure or layers 802 may contain a plurality of strip members which extend throughout the internal portion of the apparatus 800 between the layers 802 similarly to the veins of a leaf. The strip members, as described herein, may be comprised of a porous or open cell material such as foam for collecting and transporting bodily fluids which are collected by the strip members. Furthermore, the strip members may be fluidly coupled to one another such that a network of the strip members extends throughout the internal portion of the apparatus 800 and are fluidly coupled to the tubing 107 at the proximal end of the apparatus 800 where the terminal proximal end of the strip member is fluidly coupled to an opening of the tubing 107.

In the variation shown, a strip member may form a main stem portion 804 which may be fluidly coupled at its proximal end to the tubing 107 and which may extend along the longitudinal axis 816 of the apparatus 800 and may define a terminal shoot 810 of the main stem portion 804 near or at a distal end of the apparatus 800. One or more primary branch portions 806 may extend at an angle C, C′ relative to the main stem portion 804, e.g., forming an acute angle away from the proximal end of the apparatus 800. Each of the primary branch portions 806 may in turn have one or more secondary branch portions 808 extending away from its respective primary branch portion 806 at an angle D, E relative to an axis of the primary branch portion 806.

In this manner, the primary branch portions 806 and secondary branch portions 808 may “innervate” the interior of the apparatus 800 to provide fluid collection and transport throughout the apparatus 800 for removal through the main stem portion 804 and proximally out through tubing 107. Furthermore, the primary branch portions 806 and secondary branch portions 808 may be symmetrically configured to extend about the main stem portion 804 but the individual primary and second branch portions may be uniformly or arbitrarily configured to be symmetrical or asymmetrical about the main stem portion 804 in other examples.

The example shown further illustrates an apparatus 800 having four primary branch portions 806 extending at uniform distances on either side of the main stem portion 804 where each primary branch portion 806 has between one and three secondary branch portions 808 extending away from a respective primary branch portion 806. In other examples, however, any number of primary branch portions 806 may be utilized where each primary branch portion 806 may have any number of secondary branch portions 808, as practicable.

Furthermore, the primary branch portion 806 and secondary branch portions 808 may have a width W, as shown in the detail view of FIG. 14A, which may vary between portions or which may be uniform. FIG. 14C shows a side view of the apparatus 800 illustrating how the members, such as the main stem portion 804, are positioned to extend through the length within the enclosure 802 and have a thickness T. The enclosure layers are also shown as having a thickness Tl.

FIG. 15A shows the apparatus 800 and an alternative apparatus 820 which is also designed as a leaf configuration having a main stem portion 822 but having a simplified design of primary branch portions 824 which extend at an angle from the main stem portion 822.

FIGS. 15B-15D illustrate an example of how the leaf design apparatus 800 may also be configured into a compact rolled configuration for advancement into a body cavity and deployment in a minimally invasive surgery. The apparatus 800 (or apparatus 820) in its rolled configuration may be positioned within a sheath (e.g., an inner deployment sheath, as previously described) or trocar 822 and advanced through a cannula 824, as shown in FIGS. 15C-15D. After insertion, the apparatus 800 may be uncompressed or unfurled by removing the trocar 822 from the apparatus 800 so as to be deployed for placement around an organ or tissue. The deployed apparatus 800 may be left within the body, the open cavity comprising one of more flaps of tissues are closed (but not sealed as there must be means for tubing egress from the cavity to the vacuum pump) by any means known in the art such as sutures, to continue NPT in situ. When a sufficient amount of NPT is completed, the apparatus 800 of any shape may be retracted using gravity and force by tensioning or pulling the tubing 107 allowing for the apparatus 800 to collapse about the tubing 107 as it is pulled due in part to the tubing connection being located along a periphery of the apparatus 800 so that the apparatus 800 may be removed from the interior of the closed cavity. The small remaining incision is then sealed by means known to one of skill in the art. FIG. 15E shows a collapsed apparatus 100 and a collapsed leaf configured apparatus 800 also in its collapsed configuration for comparison.

FIG. 16 shows a perspective exploded view of the leaf configured apparatus 800 for a detailed view of the main stem portion 804 and individual primary stem portions 806 and secondary stem portions 808.

FIG. 17 illustrates an example of the apparatus 820 which may have an overall length LT1 of, e.g., 18.0 in., and an overall width WD1 of, e.g., 10.7 in. FIG. 18 also shows another example of the leaf configuration apparatus 800 similarly having an overall length LT2 of, e.g., 18.0 in., and an overall width WD2 of, e.g., 10.7 in. The dimensions are by no means meant to be limiting and serve as examples of the number of various types of foam strips, length and width of the foam strips and the overall apparatus as each component of the apparatus may be modified depending on surgical need. However, generally for each design, the various components retain proportionality as to their dimensions in relation to another component.

FIG. 19 shows another view of the apparatus 800 which may be sized at various dimensions depending on the overall size of the apparatus 800. The apparatus 800 may be sized in standard sizes depending upon the desired use and location within the body as well as the anatomical dimensions of the tissue region to be treated. For instance, while the apparatus 800 may be sized in any various dimensions, the apparatus 800 may be also sized in a standard large (18 in.×11 in.), medium (16 in.×9.75 in.), or small size (14 in.×8.55 in.). Of course, the standard sizes for large, medium, or small may also be varied depending upon any number of factors.

Aside from the leaf configuration and other embodiments of the apparatus described herein, the apparatus may also be shaped into other alternative configurations. Another example of such an apparatus is shown in FIGS. 20A-20B which illustrate a front perspective view of an embodiment of a pitchfork-shaped apparatus 830 (described in further detail below) wherein the apparatus 830 is in situ inside a patient undergoing, e.g., a mastectomy. Frequently, oncologic resection during a mastectomy, ipsalateral axillary lymph nodes in nearby regions such as an armpit may need to be removed. Removal of these lymph nodes and breast tissue leads to internal cavities within a patient into which bodily fluids collect. Also, there may be collection of fluids in leaky lymph ducts. Such fluid “sinks” may lead to seroma formation.

Supplementing surgical operation by using the apparatus and methods disclosed herein has a dual fold function as the NPT suction forces tissues to meld together, decreasing the space between tissues as well as removing any collected fluids.

It is contemplated that in various surgical scenarios, the apparatus and methods may be used during and for any time period after surgery, including after most of an incision has been sealed. The patient may be discharged home and after it appears that the likelihood of surgical complication such as seromas is diminished, the apparatus may be removed in an “outpatient” setting. Thus, post-surgical ease of removal is another benefit for the apparatus.

FIGS. 21A-21B are perspective views of another procedure wherein the pitchfork-shaped apparatus 830 may be positioned in situ inside a patient skull undergoing brain surgery.

FIGS. 22A-22B are perspective views of another procedure wherein the pitchfork-shaped apparatus 830 may be positioned in situ inside a patient arm undergoing surgery for a large wound.

FIG. 23A-23C illustrate embodiments of the pitchfork-shaped apparatus 830 where instead of the leaf shaped configuration, the layers of the enclosure 832 may conform to a reduced number of strip members for conforming more closely upon a tissue region to be treated. In this example, the apparatus 830 may include proximal region 838 which extends distally into one or more individual members 836. The proximal strip member 834 may extend internally in fluid contact with the tubing 107 and separate into individual strip members. The variation of FIG. 23A illustrates a three prong apparatus 830 while the embodiment of FIG. 23B illustrates an apparatus having two individual prongs 840, 842. FIG. 23C illustrates yet another embodiment of an apparatus having a single individual prong 844. While three prongs are shown in the embodiment of FIG. 23A, other variations may include more than three prongs.

FIGS. 24A-24F illustrate yet another method in which a pitchfork-shaped apparatus may be deployed into a patient during, e.g., a breast surgery or a chest surgery to treat pneumothorax. The apparatus 830 may be advanced through an incision 850, e.g., in proximity to the breast, and the apparatus 830 may be positioned upon the tissue to be treated, as shown in FIGS. 24A and 24B. Alternatively, the apparatus 830 may be advanced through a separate incision 852 for placement upon the tissue region, as shown in FIG. 24C. Once the treatment has been completed, the apparatus may be pulled or tensioned for reconfiguring into a collapsed configuration about the tubing 107, as described herein, for removal from the tissue region, as shown in FIGS. 24D and 24E. With the apparatus 830 removed, the incision 852 may be closed, as shown in FIG. 24F.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment,” “in another embodiment,” or the like are used herein, these phrases are meant to generally reference embodiment possibilities, and are not intended to limit the disclosure to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another, unless indicated otherwise.

Similarly, although example processes have been described with regard to particular operations performed in a particular sequence, numerous modifications could be applied to those processes to derive numerous alternative embodiments of the present disclosure. For example, alternative embodiments may include processes that use fewer than all of the disclosed operations, processes that use additional operations, and processes in which the individual operations disclosed herein are combined, subdivided, rearranged, or otherwise altered.

This disclosure may include descriptions of various benefits and advantages that may be provided by various embodiments. One, some, all, or different benefits or advantages may be provided by different embodiments.

In view of the wide variety of useful permutations that may be readily derived from the example embodiments described herein, this detailed description is intended to be illustrative only, and should not be taken as limiting the scope of the disclosure. What is claimed as the disclosure, therefore, are all implementations that come within the scope of the following claims, and all equivalents to such implementations.

What is claimed is:

1. An apparatus for improving post-operative recovery from surgery, comprising:
one or more pliable members having a main stem portion and one or more primary branch portions extending from the main stem portion;
one or more layers encompassing the one or more pliable members and having a curved configuration, wherein the one or more layers has a deployed configuration when positioned within a body cavity and a retracted configuration when withdrawn from the body cavity; and
a connecting tube in fluid communication with and coupled to the one or more layers at a periphery of the one or more layers, wherein the one or more layers are collapsible into the retracted configuration relative to the connecting tube when a force is applied to the connecting tube.

2. The apparatus of claim 1 wherein the main stem portion extends from a proximal end to a distal end of the one or more layers.

3. The apparatus of claim 1 wherein the one or more primary branch portions extend from the main stem portion in a symmetrical pattern about a longitudinal axis.

4. The apparatus of claim 1 further comprising one or more secondary branch portions extending from the one or more primary branch portions.

5. The apparatus of claim 1 wherein the one or more pliable members comprise porous or open cell members.

6. The apparatus of claim 1 further comprising a plurality of openings distributed throughout the one or more layers.

7. The apparatus of claim 1 wherein the one or more pliable members are fluidly connected to the connecting tube.

8. The apparatus of claim 1 further comprising a negative pressure mechanism that is in fluid communication with the one or more pliable members through the connecting tube.

9. The apparatus of claim 1 wherein the one or more layers are constructed from a material selected from the group consisting of polyethylene and polyurethane.

10. The apparatus of claim 1 wherein the one or more pliable members are constructed from a material selected from the group consisting of polyurethane (PU), polyethylene (PE) and polyvinyl alcohol (PVA) foam.

11. The apparatus of claim 1 wherein the apparatus is configured to be reconfigured into a low profile compact configuration.

12. The apparatus of claim 11 further comprising a cannula through which the apparatus is deliverable.

13. A method of treating a tissue region, comprising:
advancing a treatment apparatus in a low profile compact shape through an entry lumen into a tissue region to be treated;
reconfiguring the treatment apparatus into a deployed and expanded configuration;
positioning the treatment apparatus upon the tissue region;
applying negative vacuum pressure therapy to the treatment apparatus such that a bodily fluid is removed via the treatment apparatus; and
applying a tensioning force to a connecting tube coupled to a periphery of the treatment apparatus such that the treatment apparatus reconfigures into a collapsed configuration about the connecting tube for removal from the tissue region.

14. The method of claim 13 wherein the treatment apparatus comprises:
one or more pliable members having a main stem portion and one or more primary branch portions extending from the main stem portion;
one or more layers encompassing the one or more pliable members and having a curved configuration, wherein the one or more layers has a deployed configuration when positioned within a body cavity and a retracted configuration when withdrawn from the body cavity; and
wherein the connecting tube is in fluid communication with and coupled to the one or more layers at a periphery of the one or more layers, wherein the one or more layers are collapsible into the retracted configuration relative to the connecting tube when a force is applied to the connecting tube.

15. The method of claim 13 wherein advancing the treatment apparatus comprises advancing the treatment apparatus in a rolled configuration through the entry lumen.

16. The method of claim 13 wherein advancing the treatment apparatus comprises advancing the treatment apparatus into proximity of a breast of a patient.

17. The method of claim 13 wherein advancing the treatment apparatus comprises advancing the treatment apparatus into proximity of a brain of a patient.

18. The method of claim 13 wherein advancing the treatment apparatus comprises advancing the treatment apparatus into proximity of a wound of a patient.

19. An apparatus for improving post-operative recovery from surgery, comprising:
a pliable member having a main stem portion and at least one primary branch portion extending from the main stem portion;
one or more layers encompassing the one or more pliable members, wherein the one or more layers has a deployed configuration when positioned within a body cavity and a retracted configuration when withdrawn from the body cavity; and
a connecting tube in fluid communication with and coupled to the one or more layers at a periphery of the one or more layers, wherein the one or more layers are collapsible into the retracted configuration relative to the connecting tube when a force is applied to the connecting tube.

20. The apparatus of claim 19 wherein the main stem portion extends from a proximal end to a distal end of the one or more layers.

21. The apparatus of claim 19 wherein the one or more primary branch portions extend from the main stem portion in a symmetrical pattern about a longitudinal axis.

22. The apparatus of claim 19 wherein the one or more primary branch portions form a pitchfork-shaped configuration.

23. The apparatus of claim 19 wherein the one or more pliable members comprise porous or open cell members.

24. The apparatus of claim 19 further comprising a plurality of openings distributed throughout the one or more layers.

25. The apparatus of claim 19 wherein the one or more pliable members are fluidly connected to the connecting tube.

26. The apparatus of claim 19 further comprising a negative pressure mechanism that is in fluid communication with the one or more pliable members through the connecting tube.

27. The apparatus of claim 19 wherein the one or more layers are constructed from a material selected from the group consisting of polyethylene and polyurethane.

28. The apparatus of claim 19 wherein the one or more pliable members are constructed from a material selected from the group consisting of polyurethane (PU), polyethylene (PE) and polyvinyl alcohol (PVA) foam.

29. The apparatus of claim 19 wherein the apparatus is configured to be reconfigured into a low profile compact configuration.

30. The apparatus of claim 29 further comprising a cannula through which the apparatus is deliverable.
